(19) United States
(12) Patent Application Publication
(10) Pub. No.: US 2006/0265043 A1
(43) Pub. Date: Nov. 23, 2006

(54) Method and apparatus for treating vulnerable plaque

(76) Inventors: Evgenia Mandrusov, Santa Clara, CA (US); Deborah Kilpatrick, Los Altos, CA (US); Murthy Simhambhatla, San Jose, CA (US); Syed Faiyez Ahmed Hossainy, Fremont, CA (US); Jessica G. Chiu, Belmont, CA (US); Mina Chow, Campbell, CA (US); Eugene T. Michal, San Francisco, CA (US); Charles D. Claude, Flagstaff, AZ (US); William E. Webler, Escondido, CA (US)

Correspondence Address: James C. Scheller, Jr.;BLAKELY, SOKOLOFF, TAYLOR & ZAFMAN LLP, Seventh Floor, 12400 Wilshire Boulevard, Los Angeles, CA, 90025-1026, US

(21) Appl. No.: 11/388,355

(22) Filed: Mar. 24, 2006

Related U.S. Application Data
(63) Continuation-in-part of application No. 11/283,032, filed on Nov. 17, 2005.
Application No. 11/283,032 is a division of application No. 10/262,151, filed on Sep. 30, 2002, now Pat. No. 7,008,411.

(51) Int. Cl. A61F 2/06

(52) U.S. Cl. 623/1.11; 623/1.42

(57) ABSTRACT

Various apparatuses and methods are described to treat vulnerable plaque with the use of combination drug therapy. In one embodiment, the apparatus has an elongated catheter body adapted for insertion in a body lumen, with a drug delivery device attached near a distal portion of the elongated body. The drug delivery device is configured to deliver a therapeutic or biologically active agent to stabilize a vulnerable plaque. In another embodiment, a drug eluting stent is delivered and deployed in conjunction with a second apparatus that delivers drug in pressurized retrograde perfusion. Still another embodiment uses a uniquely shaped balloon to deploy a stent while utilizing a drug infusion needle to inject drug into the vessel wall through a hollow guide wire.

This application is a continuation-in-part of co-pending U.S. patent application Ser. No. 11/283,032, filed on Nov. 17, 2005, which is a divisional of co-pending U.S. patent application Ser. No. 10/262,151, filed on Sep. 30, 2002.

### FIELD OF THE INVENTION

The invention, in one embodiment, relates generally to the treatment of coronary disease, and more particularly, in one embodiment, to the stabilization of vulnerable plaque.

### BACKGROUND OF THE INVENTION

Coronary heart disease is generally thought to be caused by the narrowing of coronary arteries by atherosclerosis, the buildup of fatty deposits in the lining of the arteries. The process that may lead to atherosclerosis begins with the accumulation of excess fats and cholesterol in the blood. These substances infiltrate the lining of arteries, gradually increasing in size to form deposits commonly referred to as plaque or atherosclerotic occlusions. Plaques narrow the arterial lumen and impede blood flow. Blood cells may collect around the plaque, eventually creating a blood clot that may block the artery completely.

The phenomenon of “vulnerable plaque” has created new challenges in recent years for the treatment of heart disease. Unlike occlusive plaques that impede blood flow, vulnerable plaque develops within the arterial walls, but it often does so without the characteristic substantial narrowing of the arterial lumen which produces symptoms. As such, conventional methods for detecting heart disease, such as an angiogram, may not detect vulnerable plaque growth into the arterial wall. After death, an autopsy can reveal the plaque congested in arterial wall that could not have been seen otherwise with currently available medical technology.

The intrinsic histological features that may characterize a vulnerable plaque include increased lipid content, increased macrophage, foam cell and T lymphocyte content, and reduced collagen and smooth muscle cell (SMC) content. This fibroatheroma type of vulnerable plaque is often referred to as “soft,” having a large lipid pool of lipoproteins surrounded by a fibrous cap. The fibrous cap contains mostly collagen, whose reduced concentration combined with macrophage derived enzyme degradations can cause the fibrous cap of these lesions to rupture under unpredictable circumstances. When ruptured, the lipid core contents, thought to include tissue factor, contact the arterial bloodstream, causing a blood clot to form that can completely block the artery resulting in an acute coronary syndrome (ACS) event. This type of atherosclerosis is coined “vulnerable” because of unpredictable tendency of the plaque to rupture. It is thought that hemodynamic and cardiac forces, which yield circumferential stress, shear stress, and flexion stress, may cause disruption of a fibroatheroma type of vulnerable plaque. These forces may rise as the result of simple movements, such as getting out of bed in the morning, in addition to in vivo forces related to blood flow and the beating of the heart. It is thought that plaque vulnerability in fibroatheroma types is determined primarily by factors which include: (1) size and consistency of the lipid core; (2) thickness of the fibrous cap covering the lipid core; and (3) inflammation and repair within the fibrous cap.

FIG. 1A illustrates a partial cross-section of an artery having a narrowed arterial lumen caused by the presence of occlusive atherosclerosis. Plaque accumulates to impede and reduce blood flow through the arterial lumen and thus often causes symptoms (e.g., angina pectoris). The arrows indicate the direction of blood flow through the arterial lumen. FIG. 1B illustrates an occlusive atherosclerosis within an arterial lumen resulting in significant reduction in lumen patency. This type of atherosclerosis can easily be detected through current diagnostic methods such as an angiogram. FIG. 1B also illustrates, downstream from the occlusive atherosclerosis, a fibroatheroma type of vulnerable plaque. The vulnerable plaque, with a lipid core, develops mostly within the arterial wall with minimal occlusive effects such that it is not easily detected by current diagnostic methods. This is partially due to a phenomenon known as “positive remodeling,” which allows the vessel to respond to the presence of disease. The fibroatheroma vulnerable plaque has grown into the positively remodeled arterial wall so that vessel occlusion has not been manifested. A fibrous cap surrounds the vulnerable plaque.

FIGS. 2A-2C illustrate a cross-sectional view of the accumulation of vulnerable plaque in the arterial wall. FIG. 2A illustrates an arterial wall that is not affected by atherosclerosis. The normal arterial wall consists of an intima layer, a media layer, and an adventitia layer. The intima is in direct contact with the blood flow within the arterial lumen. The intima consists mainly of a monolayer of endothelial cells. The media consists mostly of smooth muscle cells and extracellular matrix proteins. The outermost layer of the arterial wall, the adventitia, is primarily collagenous and contains nerves, blood vessels, and lymph vessels. FIG. 2B illustrates the large presence of a fibroatheroma type vulnerable plaque surrounded by a fibrous cap within the arterial wall. The vulnerable plaque consists mainly of a large lipid core. The fibrous cap layer shields the lumen of the artery from the thrombogenic components in the core. FIG. 2C illustrates an occlusive thrombosis event resulting from the rupturing of the fibrous cap. Thrombogenic components in the vulnerable plaque contact luminal blood and cause the thrombotic event.

Autopsy studies and other evidence strongly suggest that the presence of a current acute coronary syndrome (ACS) event and/or existing thrombus at certain plaque sites may correlate to predicting a future ACS event in a given patient. The latter indicates the likelihood of a prior thrombotic event (e.g., fibroatheroma rupture) after which the plaque was able to heal itself, or complete occlusion of the vessel was somehow prevented. Autopsy studies also indicate that it is reasonable to expect that at least one vulnerable plaque could exist in the majority of catheterization laboratory patients being treated for arterial blockage from visible, occlusive atherosclerosis. Many of the patients at highest risk, therefore, for future ACS events may already be receiving interventional treatment, even though current methods to diagnose occlusive plaques (i.e., non-vulnerable type plaque) are not effective for enabling therapy for vulnerable plaque. Furthermore, treating both the occlusive plaques and the vulnerable plaque in one procedure might be beneficial and desirable compared to separate treatments. This would provide a greater convenience to the patient and for the physician.

Lastly, the inventions hereby disclosed generally related to the field of vascular interventional therapy. Specifically, various embodiments in this invention refer to guide wires and delivery catheters. U.S. Pat. No. 6,540,734B1 titled “Multi-Lumen Extrusion Tubing” by Jessica Chiu et al. and U.S. patent application Ser. No. 11/676,616 titled “Deflectable catheter Assembly and Method of Making Same” by Mina Chow et al. are herein incorporated by reference as art related to the current disclosure.

### SUMMARY OF THE INVENTION

An apparatus and method to treat vulnerable plaque are described. In one embodiment, the apparatus has an elongated catheter body adapted for insertion in a body lumen, with a drug delivery device attached near a distal portion of the elongated body. The drug delivery device is configured to deliver a biologically active agent to stabilize a vulnerable plaque.

### BRIEF DESCRIPTION OF THE DRAWINGS

The present invention is illustrated by way of example, and not limitation, in the figures of the accompanying drawings in which:

FIG. 1A illustrates a partial cross-section of an arterial lumen having occlusive plaque.

FIG. 1B illustrates a partial cross-section of an arterial lumen having occlusive plaque and vulnerable plaque.

FIGS. 2A-2C illustrate the vessel morphology and the rupturing of a vulnerable plaque.

FIGS. 3A-3B illustrate the stabilization a vulnerable plaque by reducing the size of the lipid core and strengthening and increasing the thickness of the fibrous cap.

FIG. 4 illustrates one embodiment of using a drug delivery stent to treat a vulnerable plaque downstream from an occlusive plaque.

FIGS. 5A-5C illustrate an alternative embodiment of using a drug delivery stent to treat a vulnerable plaque downstream from an occlusive plaque.

FIG. 6 illustrates one embodiment of microparticles released towards a vulnerable plaque.

FIG. 7 illustrates one embodiment of a stent graft used to treat a vulnerable plaque.

FIGS. 8A-8B illustrate cross-sectional views of a stent graft.

FIGS. 9A-9D illustrate various embodiments of using a needle catheter to treat a vulnerable plaque.

FIGS. 10A-10B illustrate one embodiment of a needle catheter.

FIGS. 11A-11D illustrate various methods for treating vulnerable plaque.

FIG. 12 illustrates one embodiment of inducing therapeutic angiogenesis growth near a vulnerable plaque.

FIGS. 13A-13B illustrate cross-sectional views of one embodiment of a drug eluting stent that can be used to strengthen and to increase the thickness of the fibrous cap of the vulnerable plaque in a controlled manner.

FIGS. 14A-14F illustrate the combination therapy of administering one drug via a drug eluting stent and a second drug via a venous vessel by way of retrograde perfusion.

FIGS. 15A-15C illustrate drug delivery using a balloon catheter with drug injection needles.

FIGS. 16A-16C illustrate various embodiments of a proximal end plug for a modified hollow guide wire.

FIGS. 17A-17C illustrate various views of a modified hollow guide wire.

FIGS. 18A-18C illustrate various views of a distal portion of a modified hollow guide wire.

FIGS. 19A-19C illustrate different views of a connection mechanism and control mechanism of a injection needle system and a detachable luer used to integrate an injection needle with a modified hollow guide wire.

FIGS. 20A-20B illustrate different views of a combination of a guide wire and a stent delivery system with a balloon having a groove and shaped in accordance with the teachings of the present invention.

FIGS. 21A-21C illustrate cross-sectional views of different extrusion embodiments of a balloon having a groove and shaped in accordance with the teachings of the present invention.

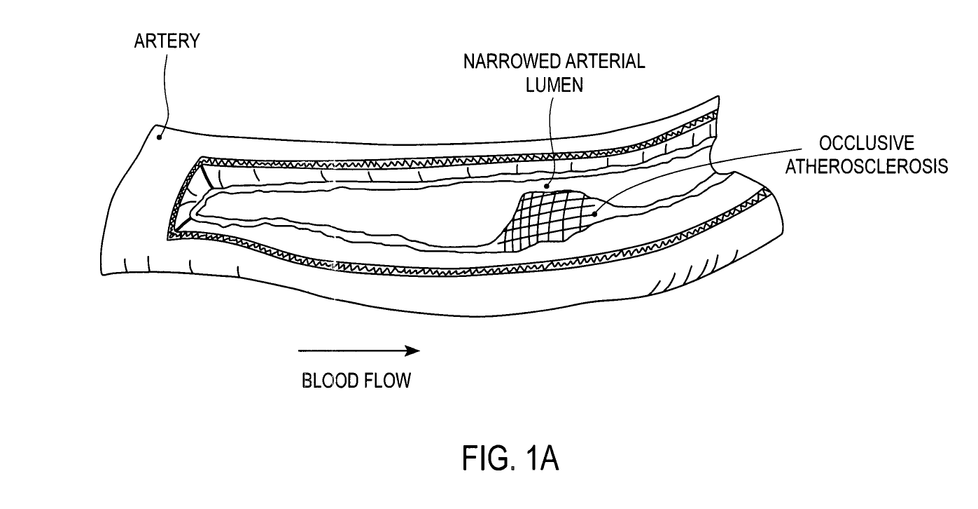

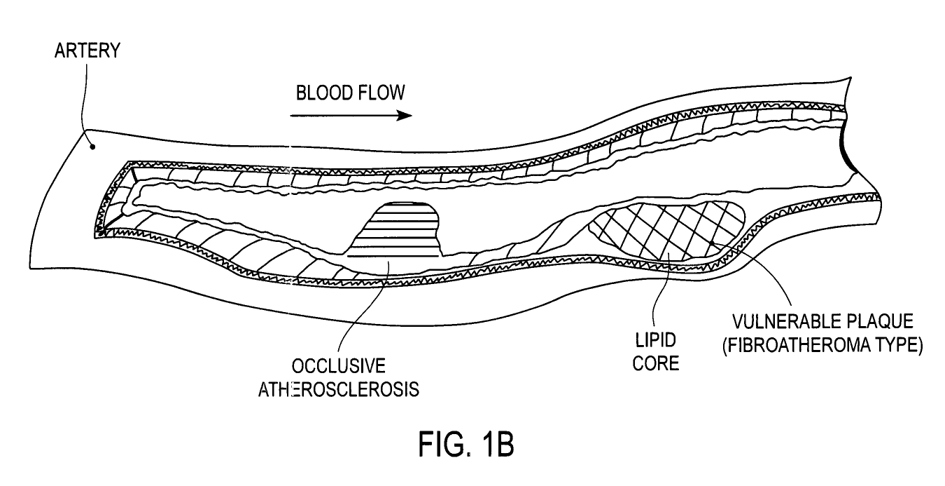

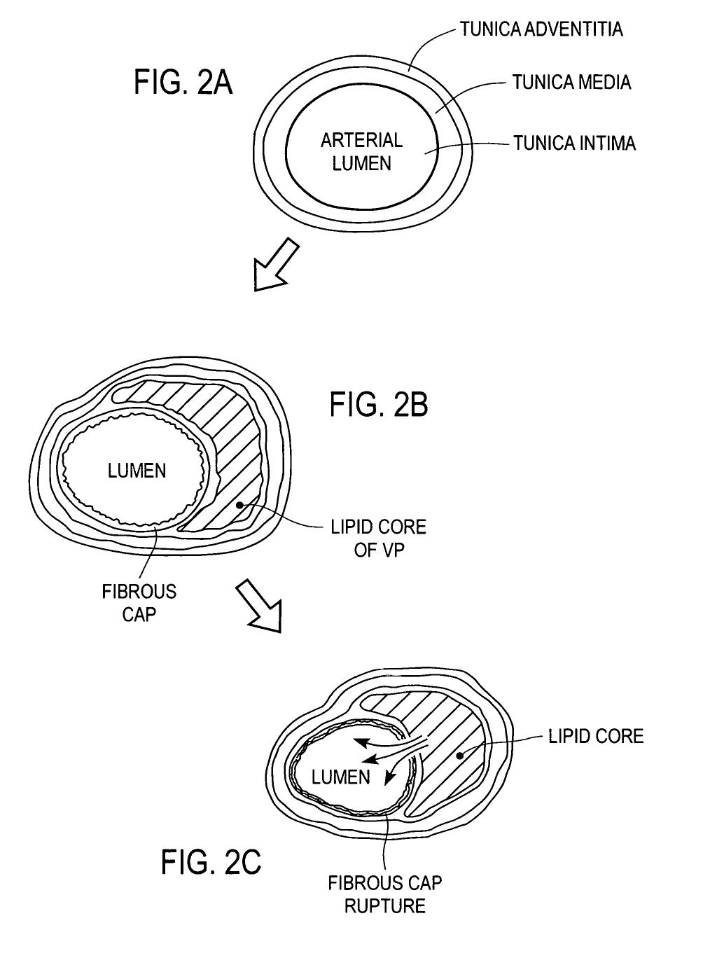

### DETAILED DESCRIPTION

In the following description, numerous specific details are set forth such as examples of specific, components, processes, etc. in order to provide a thorough understanding of various embodiment of the present invention. It will be apparent, however, to one skilled in the art that these specific details need not be employed to practice various embodiments of the present invention. In other instances, well known components or methods have not been described in detail in order to avoid unnecessarily obscuring various embodiments of the present invention. The term “coupled” as used herein means connected directly to or indirectly connected through one or more intervening components, structures or elements. The terms “drugs”, “biologically active agents”, and “therapeutic agents” are used interchangeably to refer to agents (e.g., chemical and biological substances) to treat, in one embodiment, coronary artery and related diseases including for example, atherosclerotic occlusions and vulnerable plaque. Thus, use of the term “drug” is not intended to limit the scope thereof but is intended to include biologically active agents and therapeutic agents unless otherwise indicated herein.

Apparatuses and their methods of use to treat vulnerable plaque are described. In one embodiment, the vulnerable plaque or the region of the artery containing the vulnerable plaque may be treated alone or in combination with treating occlusive atherosclerosis. The benefit is that any vulnerable, but not yet occlusive plaques would be treated without having to place a therapeutic implant (e.g., a stent) at the vulnerable plaque region. The only implant placed would be that already being used to scaffold and treat the existing occlusive plaque. In the following description, the stabilization of vulnerable plaque is described with respect to treatment within the artery. The coronary artery is just one region in the body where vulnerable plaque may form. As such, it can be appreciated that the stabilization of vulnerable plaque may be achieved in any vessel of the body where vulnerable plaque may exist.

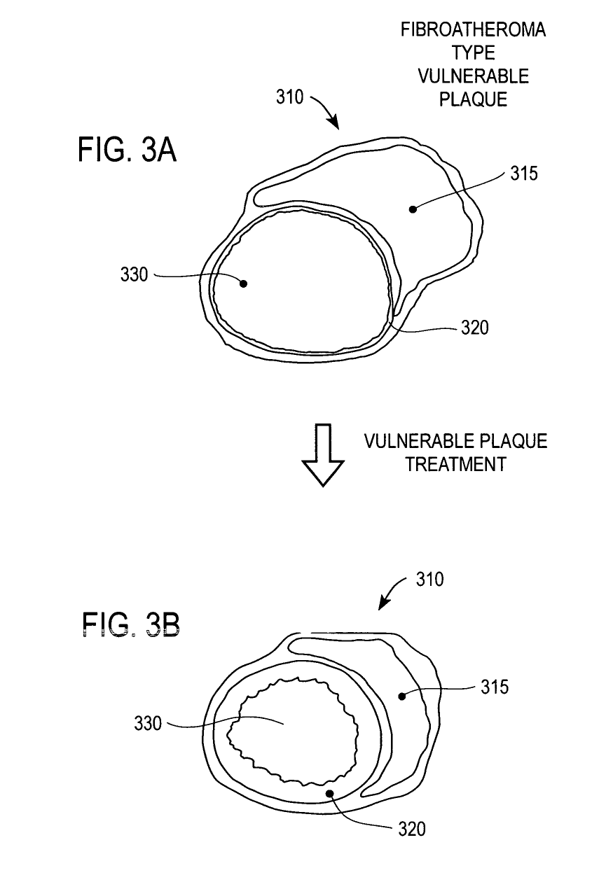

FIGS. 3A-3B illustrate a cross-sectional view of the stabilization of vulnerable plaque. FIG. 3A shows a large vulnerable plaque 310 having lipid core 315 separated from arterial lumen 330 by thin fibrous cap 320. Thin fibrous caps and reduced collagen content or degraded collagen in the fibrous caps increase a plaque's vulnerability to rupture. As illustrated in FIG. 3B, vulnerable plaque 310 has been stabilized by thickening and/or strengthening fibrous cap 320 that separates lipid core 315 from arterial lumen 330. This reduces the likelihood of fibrous cap 320 rupturing. Additionally, lipid core 315 redistribution has occurred in combination with strengthening fibrous cap 320. Vulnerable plaque 310 may also be treated by inducing collateral artery or vessel growth near the vulnerable plaque region such that, in the event of fibrous cap rupture or occlusive thrombosis, an alternative blood path exists to bypass the ruptured region (not shown).

Drug Eluting Stents

In one embodiment, a drug eluting stent may be implanted at the region of vessel occlusion that may be upstream from a vulnerable plaque region. As discussed above, autopsy studies have shown that vulnerable plaque regions commonly exist in the vicinity of occlusive plaques. A medical device, such as a drug eluting stent, may be used to treat the occlusive atherosclerosis (i.e., non-vulnerable plaque) while releasing a drug or biologically active agent to treat a vulnerable plaque region distal or downstream to the occlusive plaque. The drug may be released slowly over time, and may include for example, anti-inflammatory or anti-oxidizing agents. Biologically active agents may also be released include cells, proteins, peptides, and related entities.

The eluting stent may have the vulnerable plaque treating drug or agent dispersed on the surface of the stent, or co-dissolved in a matrix solution to be dispersed on the stent. Other methods to coat the stent with a vulnerable plaque treating drug include dip coating, spin coating, spray coating, or other coating methods commonly practiced in the art.

In one embodiment, therapeutic or biologically active agents may be released to induce therapeutic angiogenesis, which refers to the processes of causing or inducing angiogenesis and arteriogenesis, either downstream, or away from the vulnerable plaque. Arteriogenesis is the enlargement of pre-existing collateral vessels. Collateral vessels allow blood to flow from a well-perfused region of the vessel into an ischemic region (from above an occlusion to downstream from the occlusion). Angiogenesis is the promotion or causation of the formation of new blood vessels downstream from the ischemic region. Having more blood vessels (e.g., capillaries) below the occlusion may provide for less pressure drop to perfuse areas with severe narrowing caused by a thrombus. In the event that an occlusive thrombus occurs in a vulnerable plaque, the myocardium perfused by the affected artery is salvaged. Representative therapeutic or biologically active agents include, but are not limited to, proteins such as vascular endothelial growth factor (VEGF) in any of its multiple isoforms, fibroblast growth factors, monocyte chemoatractant protein 1 (MCP-1), transforming growth factor alpha (TGF-alpha), transforming growth factor beta (TGF-beta) in any of its multiple isoforms, DEL-1, insulin like growth factors (IGF), placental growth factor (PLGF), hepatocyte growth factor (HGF), prostaglandin E1 (PG-E1), prostaglandin E2 (PG-E2), tumor necrosis factor alpha (TBF-alpha), granulocyte stimulating growth factor (G-CSF), granulocyte macrophage colony-stimulating growth factor (GM-CSF), angiogenin, follistatin, and proliferin, genes encoding these proteins, cells transfected with these genes, pro-angiogenic peptides such as PR39 and PR11, and pro-angiogenic small molecules such as nicotine.

In another embodiment, therapeutic or biologically active agents to treat the vulnerable plaque may be delivered through the bloodstream or vessel wall. These therapeutic or biologically active agents include, but are not limited to, lipid lowering agents, antioxidants, extracellular matrix synthesis promoters, inhibitors of plaque inflammation and extracellular degradation, estradiol drug classes and its derivatives.

Prospective studies of high-risk patients in whom complex plaques were found have indicated that many of the ACS events can happen within six months to one year after a patient has an occlusive atherosclerosis lesion treated. In other words, there is a clinical reason to believe that it would be efficacious to try and actively treat lesions in those patients for a three to six-month period of time after treatment of occlusive atherosclerosis to prevent a recurrent ACS event. Examples of devices to treat vulnerable plaque regions include drug eluting stents, and drug loaded bioerodable and bioadhesive microparticles.

In one embodiment, the polymer may be coated on a stent using dip coating, spin coating, spray coating or other coating methods known in the art. The drug can alternatively be encapsulated in microparticles or nanoparticles and dispersed in a stent coating. A diffusion limiting top-coat may optionally be applied to the above coatings. The active agents may optionally be loaded on a stent together either by adding them together to the solution of the matrix polymer before coating, or by coating different layers, each containing a different agent or combination of agents. The drug eluting stent can alternatively have an active agent or a combination of agents dispersed in a bioerodable stent forming polymer.

Vulnerable plaque regions may also be treated independent of treating occlusive lesions near the vulnerable plaque regions. In another embodiment, a vulnerable plaque treatment drug or biologically active agent may be injected through or around the fibrous cap of a vulnerable plaque. Alternatively, in the event of a thrombotic event, a drug may be injected to prevent complete occlusion of the vessel. In one embodiment, a needle catheter may be used to inject the drug. The needle catheter may be modified to accommodate the following targets around the vulnerable plaque: fibrous cap, proteoglycan-rich surface layer, subintimal lipid core, proximal or distal regions of the plaque, media containing smooth muscle cells around the lipid core, and peri-adventitial space. In another embodiment, the needle catheter may include a sensing capability to determine penetration depth of the needle. Furthermore, the needle catheter may be configured to adopt balloons of various sizes to control the angle of needle penetration. Moreover, the use of balloons would enable accurate penetration of the needle at the desired target.

In another embodiment, a drug eluting stent may be used to strengthen or increase the thickness of the fibrous cap of the vulnerable plaque in a controlled manner. Increasing the thickness of the fibrous cap may redistribute and lower the stresses in the fibrous cap. This may stabilize the plaque and prevent it from rupturing.

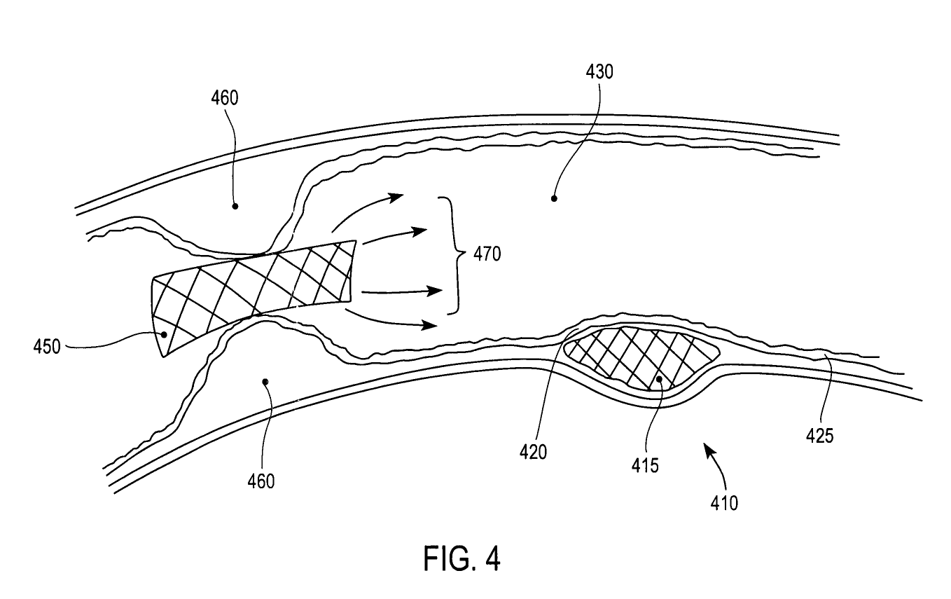

Referring to FIG. 4, a drug delivery stent 450 to treat a vulnerable plaque region is illustrated. Stent 450 is disposed in an arterial lumen 430 to treat both occlusive plaque 460 and vulnerable plaque 410 located downstream from occlusive plaque 460. As illustrated, stent 450 releases a drug (indicated by arrows 470) to treat the vulnerable plaque 410. As discussed above, vulnerable plaque regions commonly exist near occlusive plaque, and treating both might be advantageous over separate procedures. Occlusive plaque 460 has grown to cause a narrowing of the arterial lumen 430. Stent 450 is shown in a state before expansion to enlarge the diameter of the arterial lumen 430. A dilation balloon (not shown) may be used to expand stent 450, or stent 450 may be made of a material that self-expands (e.g., Nitinol) so that a dilation balloon is not needed.

As illustrated in FIG. 4, vulnerable plaque 410 is located downstream of the occlusive plaque 460 but does not show any vessel occlusion. Vulnerable plaque 410 has soft lipid core 415 with fibrous cap 420 separating vulnerable plaque 410 from arterial lumen 430. As indicated by arrows 470, stent 450 releases a drug or biologically active agent through the bloodstream of arterial lumen 430 to treat vulnerable plaque 410. In one embodiment, lipid lowering agents may be released. Lowering of serum LDL cholesterol may lead to a reduction in the amount of cholesterol entering vulnerable plaque 410, and increases high density lipoprotein (HDL) cholesterol which may contribute to active LDL removal from the vessel wall 425. Animal studies have shown that removal of lipid increases the relative collagen content of fibrous cap 420 and could increase the production of collagen, favoring vulnerable plaque stabilization. Lipid lowering animal studies suggest this also treats vulnerable plaque 410 by reducing local inflammation and the expression and activity of matrix-degrading enzymes, favoring collagen accumulation in fibrous cap 420, making it more resistant to rupture. Lipid lowering agents may also change the composition of lipid core 415 to promote plaque stabilization. It is thought that the lipid lowering agents may convert the high concentration of cholesterol esters to insoluble cholesterol monohydrate crystals, resulting in a more stiff lipid core 415 that is more resistant to plaque rupture. Lipid lowering agents include, but are not limited to hydroxy-methylglutaryl coenzyme A (HMG CoA) reductase inhibitors, niacin, bile acid resins, and fibrates.

Examples of doses of agents which may be used with embodiments of the invention, such as a drug delivery stent (i.e., the stent having been loaded with a drug which is eluted/released over time or a needle catheter) are described herein. The particular effective dose may be modified based on therapeutic results, and the following exemplary doses are acceptable initial levels which may be modified based on therapeutic results.

In an alternative embodiment, antioxidants may be released from stent 450. The oxidation of LDL cholesterol appears to have negative impact upon vessel processes during atherogenesis. Oxidized LDL binds to cell receptors on macrophages and contributes to foam cell formation. As such, antioxidants, through their inhibition of LDL oxidation, may contribute to plaque stabilization. Antioxidants may also promote plaque stabilization by reducing matrix degradation within vulnerable plaque 410. Examples of antioxidants include, but are not limited to vitamin E (α-tocopherol), vitamin C, and β-carotene supplements. Additionally, HMG CoA reductase inhibitors may also reduce oxidized LDL levels by increasing the total antioxidant capacity of plasma.

Lipid lowering agents such as statins and antioxidants may be administered at a level of about 0.5 mg/kg per day; higher doses (e.g., 5 times higher) appear to inhibit angiogenesis. See Weis et al., Statins Have Biphasic Effects on Angiogenesis, Circulation, 105(6):739-745 (Feb. 12, 2000). This dosage level may be achieved by loading a stent with about 10-600 μg of the statin, where the stent is designed to elute the statin over a period of 8 weeks. In one embodiment, the stent may have a length of 13 mm and a diameter of 3 mm. In one embodiment, the stent may have a drug release rate of 160 μg over 10 hours, or 15 μg per hour. In another embodiment, the stent may have a lower release rate of about 20 μg over 10 hours, over 2 μg per hour. Additionally, a compound called “AGI-1067”, developed by AtheroGenics, Inc. of Alpharetta, Ga., may be loaded onto the stent. AGI-1067 has been shown in studies to have direct anti-atherosclerotic effect on coronary blood vessels, consistent with reversing the progression of coronary artery disease.

In an alternative embodiment, extracellular matrix synthesis promoters may be released from stent 450. Reduced collagen content in fibrous cap 420 may result from decreased synthesis of extracellular matrix by smooth muscle cells (SMC) and/or increased breakdown by matrix-degrading proteases, thereby leading to thinning and weakening of fibrous cap 420, predisposing vulnerable plaque 410 to rupture with hemodynamic or mechanical stresses.

Vascular SMC synthesize both collagenous and noncollagenous portions of the extracellular matrix. Lack of sufficient SMC to secrete and organize the matrix in response to mechanical stress could render fibrous cap 420 more vulnerable to weakening by extracellular matrix degradation. Atherosclerosis and arterial injury lead to increased synthesis of many matrix components. In contrast, vulnerable plaque, in general, lacks a sufficient quantity of healthy matrix to provide strength to the fibrous cap to prevent rupture. Thus, promotion of SMC proliferation may lead to plaque stabilization. Delivery of cytokines and growth factors may also achieve SMC proliferation. SMC promoters and proliferative agents such as lysophosphatidic acid may be loaded onto a stent for delivery within a vessel. See Adolfsson et al., Lysophosphatidic Acid Stimulates Proliferation of Cultured Smooth Muscle Cells from Human BPH Tissue: Sildenafil and PaPaverin Generate Inhibition, Prostate, 51(1):50-8 (Apr. 1, 2002). For example, a SMC promoter may be administered at a level of about 0.5 mg/kg per day to higher doses of about 2.5 mg/kg per day. This dosage level may be achieved by loading a stent with about 10-600 μg of the SMC promoter, where the stent is designed to elute the drug over a period of 8 weeks. In one embodiment, the stent may have a drug release rate of 160 μg over 10 hours, or 15 μg per hour. In another embodiment, the stent may have a lower release rate of about 20 μg over 10 hours, over 2 μg per hour.

In an alternative embodiment, inhibitors of plaque inflammation and extracellular matrix degradation may be released from stent 450. Increased matrix degrading activity associated with enzymes derived from cells such as vascular SMC, macrophages and T lymphocytes is a common finding in vulnerable plaque. Studies suggest that matrix metalloproteinases (MMPs) are involved in matrix degradation. Plaque stabilization could be achieved through inhibition of extracellular matrix degradation by preventing the accumulation of macrophages and T lymphocytes in the vulnerable plaque or by inhibiting the proteolytic enzyme cascade directly. Possible methods to achieve MMP inhibition include increasing the levels of natural inhibitors (TIMPs) either by exogenous administration of recombinant TIMPs or administrating synthetic inhibitors. Synthetic inhibitors of MMPs, including tretracycline-derived antibiotics, anthracyclines and synthetic peptides may also be used. MMP inhibitors may be themselves antioxidants and statins based on preclinical animal data. Studies have shown MMP inhibitors, such as cerivastatin to significantly reduce tissue levels of both total and active MMP-9 in a concentration-dependent manner. See Nagashima et al., A 3-hydroxy-3-methylglutaryl Coenzyme A Reductase Inhibitor, Cerivastatin, Supresses Production of Matrix Metalloproteinase-9 in Human Abdominal Aortic Aneurysm Wall, J. Vascular Surgery, 36(1);158-63 (July 2002). As with statins as described above, MMP inhibitors may be administered at a level of about 0.5 mg/kg per day to higher doses of about 2.5 mg/kg per day. This dosage level may be achieved by loading a stent with about 10-600 μg of the SMC promoter, where the stent is designed to elute the drug over a period of 8 weeks. In one embodiment, the stent may have a drug release rate of 160 μg over 10 hours, or 15 μg per hour. In another embodiment, the stent may have a lower release rate of about 20 μg over 10 hours, over 2 μg per hour. Additionally, Avasimibe , an ACAT (Acyl-CoA: cholesterol acyltransferase) inhibitor, in the 10 mg/kg range appears to impact MMPs and plaque burden, as well as monocyte adhesion. See Rodriguez and Usher, Anti-atherogenic Effects of the acyl-CoA: Cholesterol Acyltransferase Inhibitor, Avasimibe (Cl-1011), in Cultured Primary Human Macrophages, Atherosclerosis, 161(1); 45-54 (March 2002).

Dosages and concentrations described above are exemplary, and other dosages may be applied such that when delivered over a biologically relevant time at the appropriate release rate, gives a biologically relevant concentration. The biologically relevant time may depend on the biologic target but may range from several hours to several weeks with the most important times being from 1 day to 42 days. Dosages may also be determined by conducting preliminary animal studies and generating a dose response curve. Maximum concentration in the dose response curve could be determined by the solubility of a particular compound or agent in the solution and similarly for coating a stent.

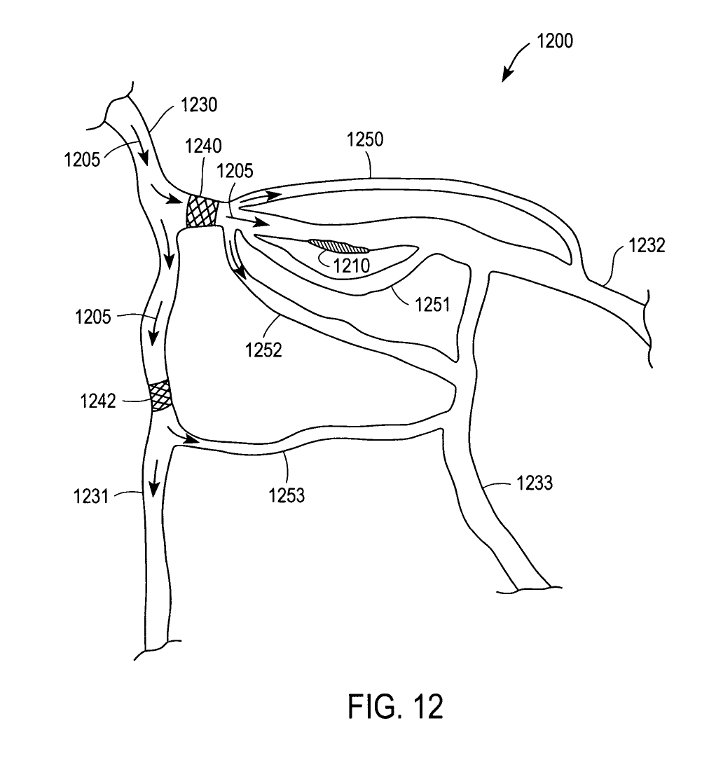

In yet another alternative embodiment, the active agent may induce collateral artery or vessel growth (i.e., angiogenesis or arteriogenesis) near the vulnerable plaque region such that, in the event of a plaque rupture and subsequent occlusive thrombosis, secondary blood paths may bypass the ruptured region and allow for continued blood flow throughout the artery. FIG. 12 illustrates one embodiment of arterial section 1200 with collateral vessels that have been induced with an active agent. Collateral vessels 1250, 1251, 1252 and 1253 provide paths for blood flow to continue through arterial section 1200 either temporarily until the occlusion is treated, or permanently to provide greater blood flow. The active agent has been delivered through drug delivery stents 1240 and 1242. Primary artery 1230 branches into sections 1231, 1232, and 1233 and arrows 1205 indicate the direction of blood flow through arterial section 1200. Vulnerable plaque 1210 is disposed within arterial branch 1232. Stent 1240 induces the growth of collateral vessels 1250, 1251 and 1252 around vulnerable plaque 1210. Collateral vessel 1251 starts upstream(near stent 1240) from vulnerable plaque 1210 and ends just downstream from vulnerable plaque 1210. Collateral vessel 1250 starts upstream from vulnerable plaque 1210 and ends further downstream of arterial branch 1232. Collateral vessel 1252 starts upstream from vulnerable plaque 1210 and ends at arterial branch 1233.

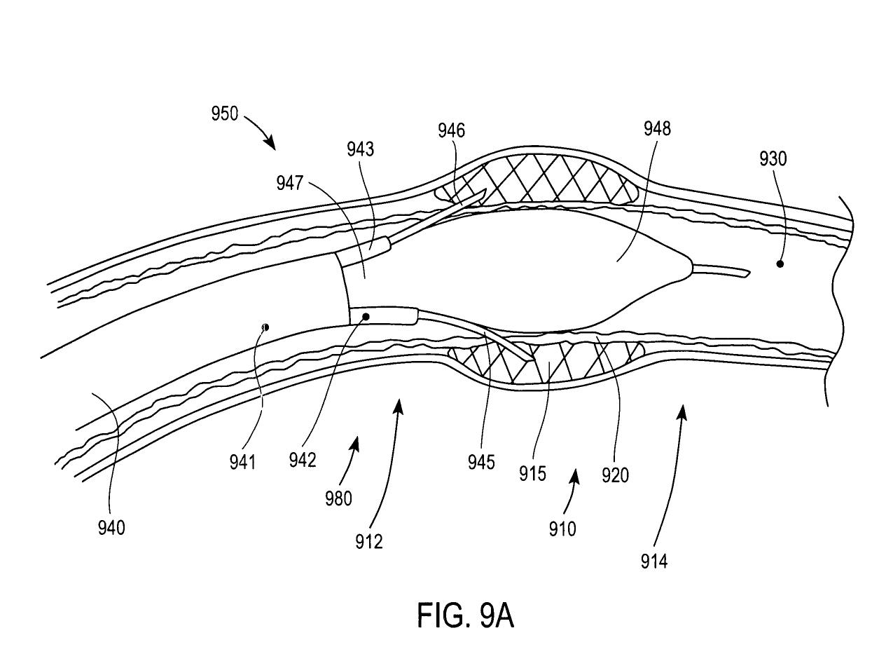

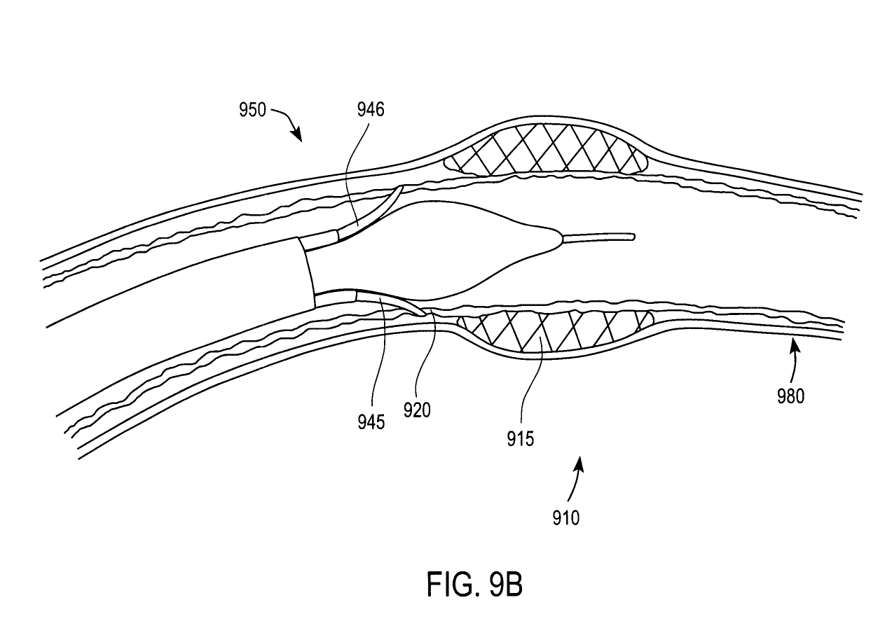

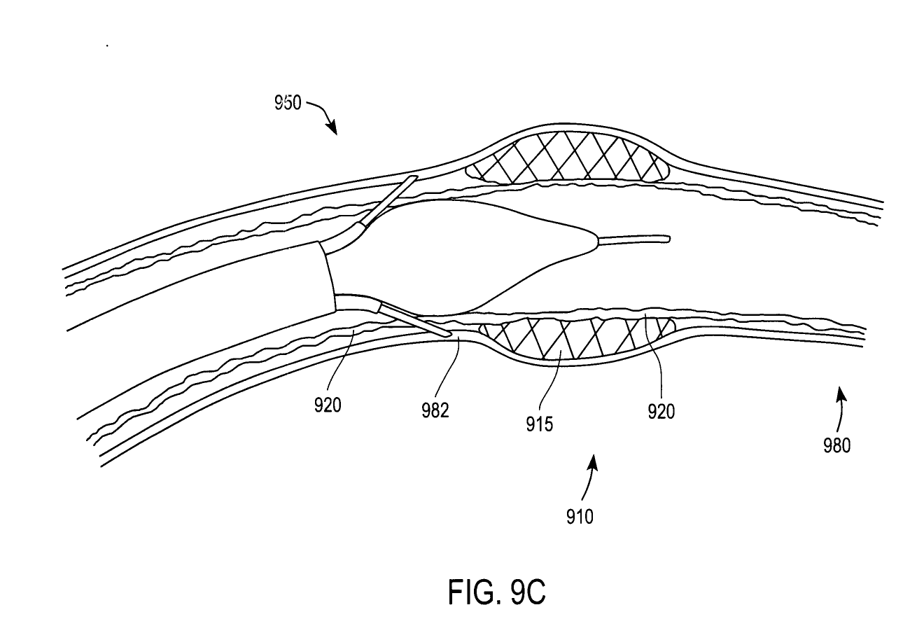

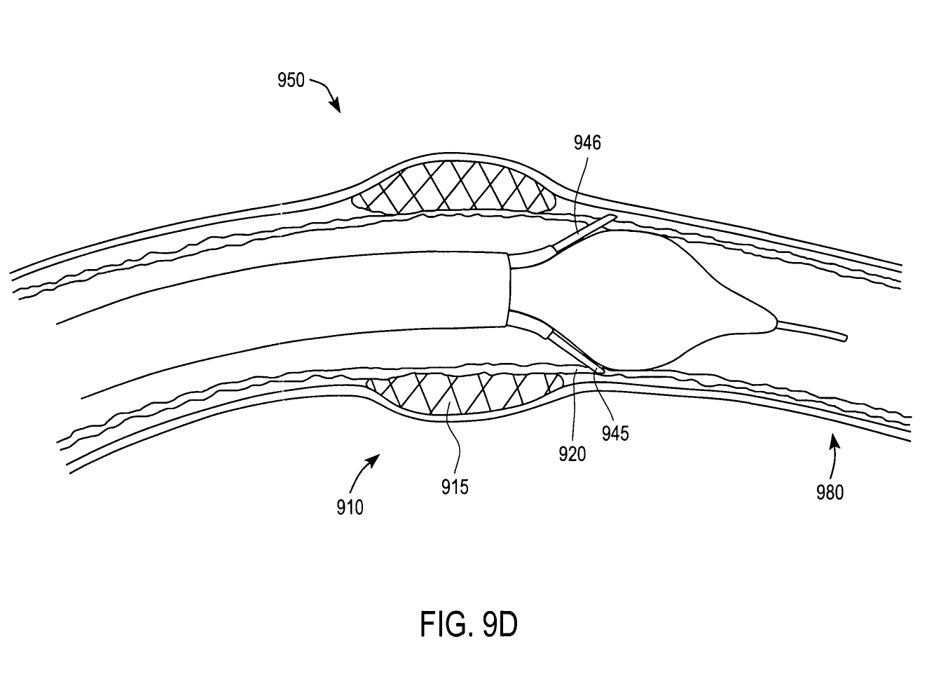

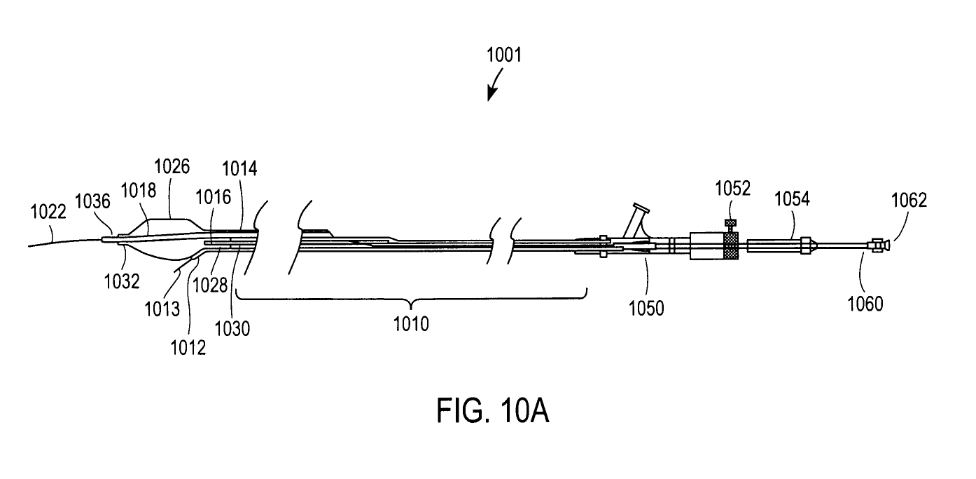

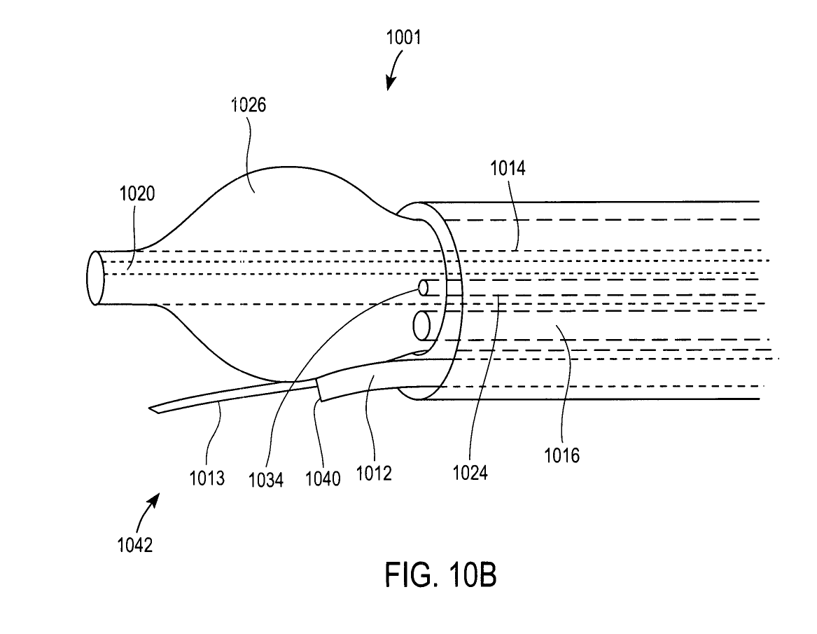

Alternatively, collateral vessel growth may be induced from an arterial branch that does not contain a vulnerable plaque. Stent 1242 carrying an active agent is disposed within arterial branch 1231 which induces collateral vessel 1253 from arterial branch 1231 to branch 1233. As such, collateral vessel 1253 may provide an alternate pathway for continued blood flow in the event vulnerable plaque 1210 ruptures. Although therapeutic or biologically active agents for angiogenesis and arteriogenesis have been described above with respect to drug eluting stents, other types of medical devices may be utilized. In one embodiment, for example, needle catheters may be used to deliver agents to induce angiogenesis and/or arteriogenesis. Needle catheters are described in greater detail below with respect to FIGS. 9-10.

In one embodiment, therapeutic or biologically active agents may be released to induce arteriogenesis or angiogenesis either downstream, or away from the vulnerable plaque to the myocardium. In the event that an occlusive thrombus occurs from a vulnerable plaque, the myocardium perfused by the affected artery may be salvaged. Representative therapeutic or biologically active agents include, but are not limited to, proteins such as vascular endothelial growth factor (VEGF) in any of its multiple isoforms, fibroblast growth factors, monocyte chemoatractant protein 1 (MCP-1), transforming growth factor alpha (TGF-alpha), transforming growth factor beta (TGF-beta) in any of its multiple isoforms, DEL-1, insulin like growth factors (IGF), placental growth factor (PLGF), hepatocyte growth factor (HGF), prostaglandin E1 (PG-E1), prostaglandin E2 (PG-E2), tumor necrosis factor alpha (TBF-alpha), granulocyte stimulating growth factor (G-CSF), granulocyte macrophage colony-stimulating growth factor (GM-CSF), angiogenin, follistatin, and proliferin, genes encoding these proteins, cells transfected with these genes, pro-angiogenic peptides such as PR39 and PR11, and pro-angiogenic small molecules such as nicotine. In one embodiment, 10-600 μg of one or a mixture of these agents may be loaded onto a stent for delivery within a vessel. These agents may have a release rate for up to eight weeks. In another embodiment, a stent may be loaded with 300 micrograms of an angiogenic agent with a release rate of eight weeks. Alternatively, a dose may be determined by those skilled in the art by conducting preliminary animal studies and generating a dose response curve. Maximum concentration in the dose response curve would be determined by the solubility of the compound in the solution.

In using drug eluting stents and related technology to deliver the vulnerable plaque treatment agent (e.g., stent 450 of FIG. 4), the active agent may be dispersed or co-dissolved directly in a solution of a matrix such as ethylene vinyl alcohol, ethylene vinyl acetate, poly(hydroxyvalerate), poly (L-lactic acid), poly(D,L-lactic acid), poly(glycolic acid), poly(lactide-co-glycolide) polycaprolactone, polyanhydride, polydiaxanone, polyorthoester, polyamino acids, poly(trimethylene carbobnate), or other suitable synthetic polymers. The polymer may be coated on a stent using dip coating, spin coating, spray coating or other coating methods known in the art.

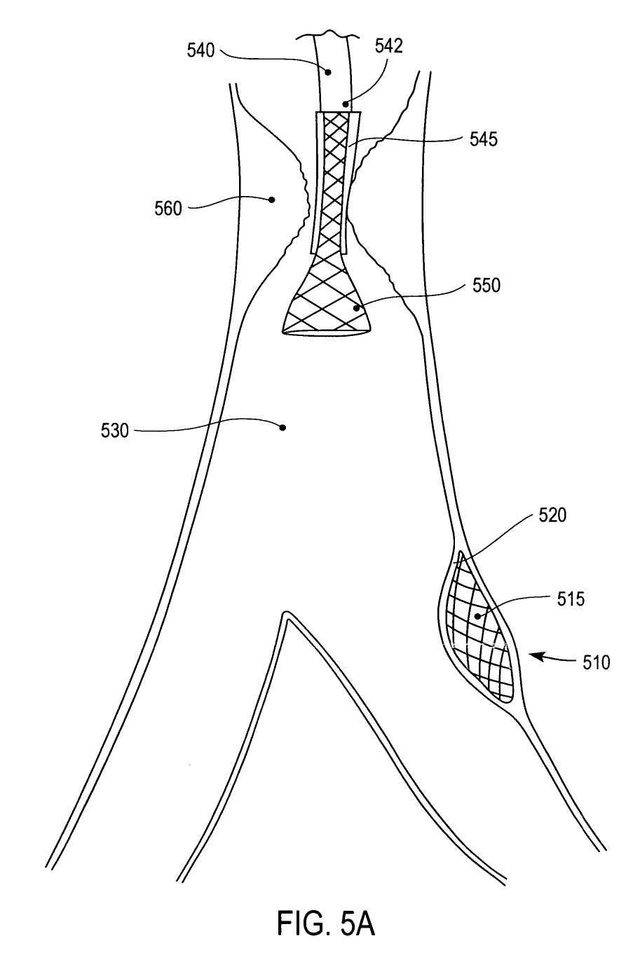

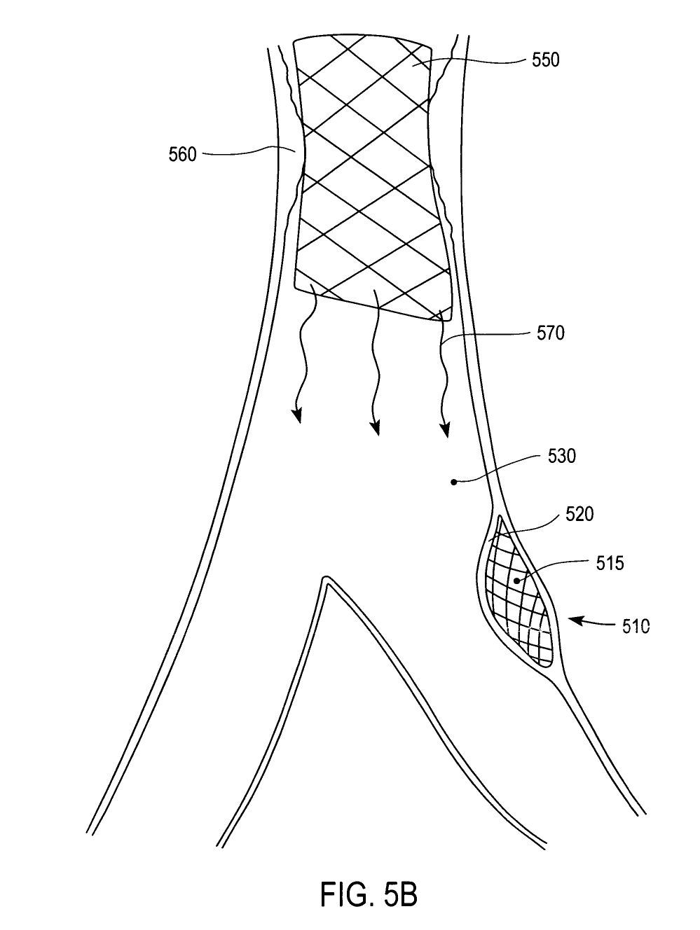

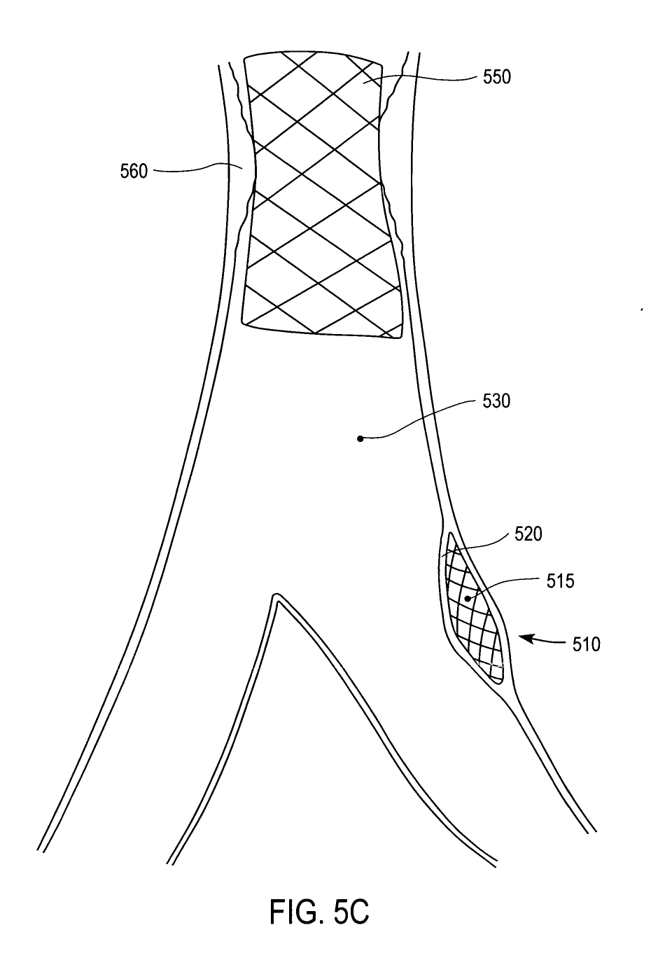

FIGS. 5A-5C illustrate the placement of drug delivery stent 550 to treat both occlusive plaque 560 and vulnerable plaque 510 localized downstream from occlusive plaque 560. In this example, vulnerable plaque 510 is located near a branched region of arterial lumen 530. In one embodiment, stent 550 is a self-expanding stent, and is disposed near distal end 542 of catheter 540. Catheter 540 is advanced through arterial lumen 530 and positioned near occlusive plaque 560. Retractable sheath 545 maintains stent 550 in a crimped and collapsed position so that stent 550 may be fit within arterial lumen 530. As illustrated in FIG. 5A, as sheath 545 retracts, stent 550 expands and applies physical pressure to occlusive plaque 560. In effect, stent 550 widens arterial lumen 530 that has been narrowed because of occlusive plaque 560. FIG. 5B illustrates stent 550 in a fully expanded position, allowing normal blood flow through arterial lumen 530.

Stent 550 may be coated with a drug or biologically active agent that releases from the surface of stent 550 when sheath 545 retracts and stent 550 becomes exposed to the blood in arterial lumen 530. The flow of the blood through arterial lumen 530 migrates the agent (as indicated by the arrows 570) towards vulnerable plaque 510. The agent targets vulnerable plaque 510. In one embodiment, the agent thickens and/or strengthens fibrous cap 520. In doing so, the likelihood of fibrous cap 520 rupturing is reduced. In another embodiment, the distribution, size or consistency of lipid core 515 is altered. A combination of agents may be utilized both to thicken fibrous cap 520 and alter the size or consistency of lipid core 515 of vulnerable plaque 510 to strengthen fibrous cap 520. FIG. 5C illustrates the treatment effects of deploying stent 550. Occlusive plaque 560 has been treated physically by compressing it against the arterial wall. Vulnerable plaque 510 has been treated through strengthening and/or thickening fibrous cap 520 and the favorable alteration of the size or distribution of the lipid core 515.

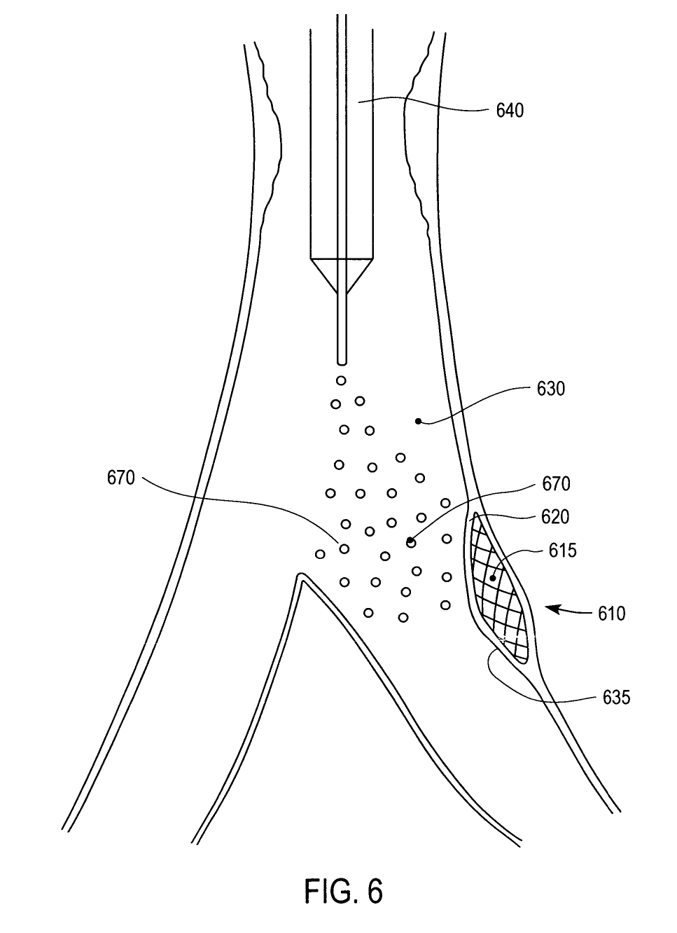

A vulnerable plaque treatment agent may be delivered independent of treating occlusive atherosclerosis. FIG. 6 illustrates a vulnerable plaque treatment agent delivered in the form of a microcapsule or microparticle 670. The use of microparticle 670 allows for delivery of a treatment agent in a controlled manner to ensure treatment over a desired period of time. Some microparticles 670 possess the characteristic of being degradable at a designated rate.

Microparticles 670 may also be designed to adhere to vessel wall 635 by blending in or coating microparticles 670 with materials that promote adhesion to vessel wall 635. Microparticles 670 may be rendered bioadhesive by modifying them with bioadhesive materials such as gelatin, hydroxypropyl methylcellulose, polymethacrylate derivatives, sodium carboxymethycellulose, monomeric cyanoacrylate, polyacrylic acid, chitosan, hyaluronic acid, anhydride oligomers, polyycarbophils, water-insoluble metal oxides and hydroxides, including oxides of calcium, iron, copper and zinc. Microparticles 670 may be modified by adsorbing the bioadhesive material on microparticles 670 through ionic interactions, coating the bioadhesive material on the microparticles by dip or spray coating, conjugating the bioadhesive material to the polymer constituting microparticle 670, or blending in the bioadhesive material into the polymer constituting the microparticles 670, before the microparticles 670 are formed.

The particle size of microcapsules 670 may be less than about 10 microns to prevent possible entrapment in the distal capillary bed. Microparticles 670 may be delivered intra-arterially near the site of vulnerable plaque 610, and also prophylactically at locations that are proximal and distal to vulnerable plaque 610 (not shown). Upon delivery with infusion catheter 640, microparticles 670 travel a short distance distally before adhering to vessel wall 630 near vulnerable plaque 610. The active agent of microparticles 670 is then released over time to thicken and/or strengthen fibrous cap 620, alter the size or distribution of lipid core 615, or both. Microparticles 670 may be delivered with infusion catheter 640 or any other delivery device known in the art. In one embodiment, infusion catheter may be a needle catheter having one or more injection ports to release microparticles 670.

Suitable polymers for the controlled-release microparticles 670 include, but are not limited to, poly (L-lactide), poly (D,L-lactide, poly(glycolide), poly lactide-co-glycolide), polycaprolactone, polyanhydride, polydiaxanone, polyorthoesters, polyamino acids, poly (trimethylene carbonate), and combinations thereof. Several methods exist for forming microparticles 670, including, but not limited to solvent evaporation, coacervation, spray drying, and cryogenic processing.

In solvent evaporation, the polymer is dissolved in a volatile organic solvent such as methylene chloride. The treatment agent is then added to the polymer solution either as an aqueous solution containing an emulsifying agent such as PVA, or as a solid dispersion, and stirred, homogenized or sonicated to create a primary emulsion of treatment agent in the polymer phase. This emulsion is stirred with an aqueous solution that contains a polymer in the aqueous phase. This emulsion is stirred in excess water, optionally under vacuum to remove the organic solvent and harden the microparticles. The hardened microparticles are collected by filtration or centrifugation and lyophilized.

The microparticles may also be formed by coacervation. In this method, a primary emulsion of treatment agent in an aqueous phase is formed as in the solvent evaporation method. This emulsion is then stirred with a non-solvent for the polymer, such as silicone oil to extract the organic solvent and form embryonic microparticles of polymer with trapped treatment agent. The non-solvent is then removed by the addition of a volatile second non-solvent such as a heptane, and the microparticles harden. The hardened microparticles are collected by filtration or centrifugation and lyophilized.

In spray drying, the treatment agent, formulated as lyophilized powder is suspended in a polymer phase consisting of polymer dissolved in a volatile organic solvent such as methylene chloride. The suspension then spray dried to produce polymer microparticles with entrapped treatment agent.

Microparticles may also be formed by cryogenic processing. In this method, the treatment agent, formulated as lyophilized powder is suspended in a polymer phase consisting of polymer dissolved in a volatile organic solvent such as methylene chloride. The suspension is sprayed into a container containing frozen ethanol overlaid with liquid nitrogen. The system is then warmed to −70° C. to liquefy the ethanol and extract the organic solvent from the microparticles. The hardened microparticles are collected by filtration or centrifugation and lyophilized.

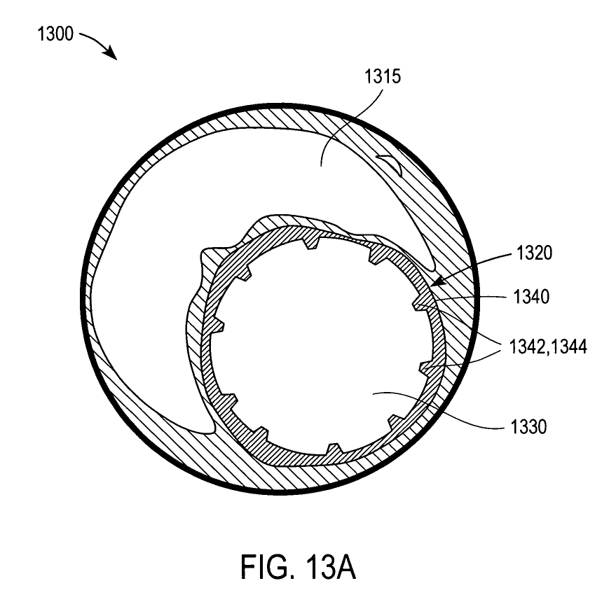

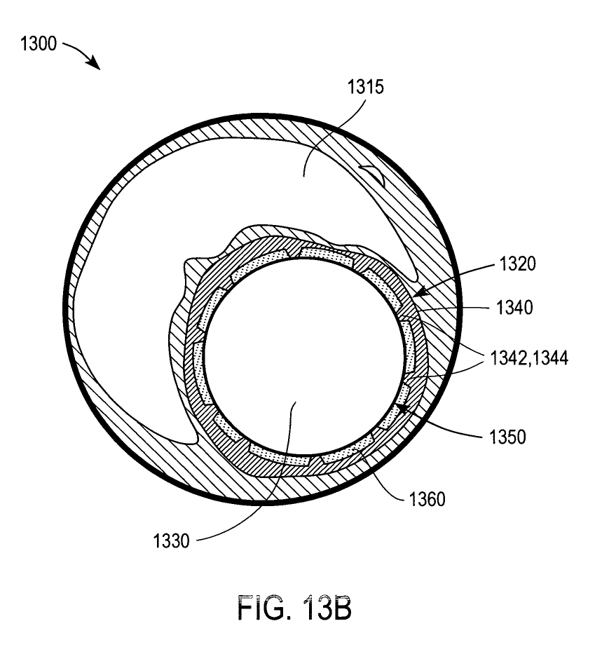

FIGS. 13A-13B illustrate cross-sectional views of one embodiment of a drug eluting stent that may be used to increase the thickness or strengthen, in a controlled manner, the fibrous cap near a vulnerable plaque. Strengthening of and increasing the thickness of the fibrous cap may redistribute and lower the stresses in the fibrous cap, effectively stabilizing the plaque and preventing its rupture.

Cross-sectional views 1300 include lumen 1330 (e.g., an arterial lumen) with lipid core 1315 of a vulnerable plaque and fibrous cap 1320. Stent 1340 having stent struts, for example struts 1342, 1344, is placed within lumen 1330 near lipid core 1315 and fibrous cap 1320. In one embodiment of using a drug eluting stent, stent 1340 serves as a vehicle for delivering an appropriate therapeutic or biologically active agent to the site of the vulnerable plaque. After stent 1340 has been deployed at a desired location, it may cause platelet deposition, fibrosis and neointimal formation in the stented region. This fibromuscular response may cause the original fibrous cap 1320 thickness to increase, thereby lowering the stresses in fibrous cap 1320 (as illustrated in FIG. 13B). This additional hyperplasia, combined with original fibrous cap 1320 produced by stent 1340 can be thought of as a “neo-cap.” Neo-cap 1360, as illustrated in FIG. 13B, has developed near the inner diameter of stent 1340. The controlled release of a drug or biologically active agent from stent 1340 may allow an increase in fibrous cap 1320 thickness because of the injury sufficient to stabilize lipid core 1315, but may minimize or prevent excessive restenosis. The type of biologically active agent, the dosage, release rate and the duration of release may influence the growth of neo-cap 1360. Therefore, by controlling these factors the growth of neo-cap 1360 may be controlled. After the thickness of fibrous cap 1320 has been increased and lipid core 1315 has been stabilized, the size of lumen 1330 may be increased by balloon angioplasty if necessary.

The biologically active agent used for controlling fibrous cap 1320 growth may be delivered using a metal stent platform (e.g., stent 1340). The drug may be released through a polymer membrane-matrix system that is deposited on the surface of the stent. Polymers such as EVAL can be used for the membrane-matrix system. Several choices of metals are available for making the stent, including but not limited to, stainless steel, cobalt-chromium alloy and shape-memory alloys such as Nitinol. Depending on the design of the stent and delivery system, it may be possible to direct the biologically active agent to act in specific locations of interest in the vulnerable plaque. For example, biologically active agents which are anti-inflammatory in nature may be optimally delivered into or around the plaque shoulder regions, a site of inflammatory cell accumulation where the lipid core edges meet the normal wall opposite the vulnerable plaque. Conversely, it may be possible to direct the biologically active agent away from specific locations of interest in the vulnerable plaque. For example, biologically active agents which are anti-restenotic, such as Actinomyocin-D, may be directed to act away from the expected regions of high stress in fibrous cap 1320, which cover lipid core 1315 in general. These regions would be the shoulder regions or the portion of fibrous cap 1320 centered circumferentially along lipid core 1315 edge nearest lumen 1330. And finally, it may also be possible to design stent 1340 or other types of delivery systems that selectively diffuse a biologically active agent appropriately, by leveraging through stent 1340 design the stress-assisted diffusion properties at the stent-plaque interface in these select regions.

The biologically active agent may also be delivered using a biodegradable polymeric stent. In this case, after the biologically active agent has eluted from the stent, the stent degrades within a certain period of time leaving behind a stabilized plaque. The polymers available for making the stent include poly-L-lactide, polyglycolic/poly-L-lactic acid (PGLA), Poly-L-latic acid (PLLA), poly-L-lactide, polycaprolactone (PCL), poly-(hydroxybutyrate/hydroxyvalerate) copolymer (PHBV) or shape memory polymers such as a compound of oligo(e-caprolactone)dimethacrylate and n-butylacrylate.

Examples of therapeutic or biologically active agents include but are not limited to rapamycin, actinomycin D (ActD) and their derivatives, antiproliferative substances, antineoplatic, antinflammatory, antiplatelet, anticoagulant, antifebrin, antithrombin, antimitotic, antibiotic and antioxidant substances. Examples of antineoplastics include taxol (paclitaxel and docetaxel). Examples of antiplatelets, anticoagulants, antifibrins and antithrombins include sodium heparin, low molecular weight heparin, hirudin, IIb/IIIa platelet membrane receptor antagonist and recombinant hirudin. Examples of antimitotic agents include methotrexate, azathioprine, vincristine, vinblastine, fluororacil, adriamycin and mutamycin. Examples of cytostatic or antiproliferative agents include angiopeptin, calcium channel blockers (such as Nifedipine), Lovastatin (an inhibitor of HMG-COA reductase, a cholestrol lowering drug from Merck). Other therapeutic or biologically active agents which may be utilized include alpha-interferon, genetically engineered epithelial cells and dexamethasone. Dosages comparable to that described above with respect to drug eluting stents may be used.

Stent Grafts

In one embodiment, a stent graft may be used for the treatment of vulnerable plaque. The stent graft may have a thin, expandable polytetrafluoroethylene (ePTFE) cylindrical tube affixed to an inner surface of a self-expandable stent. The inner surface of the ePTFE tube may have a layer of endothelial cells. The endothelial cells, when dispersed near the vulnerable plaque region, may promote cell migration to form a fully lined monolayer on the lumen surface. The stent graft may also shield existing vulnerable plaque from the possibility of an acute, thrombotic event. If the plaque ruptures, a cascade of blood-vessel wall interactions occurs, resulting in thrombosis and ultimately partial or total arterial occlusion. Therefore, shielding vulnerable plaque from the vessel lumen would eliminate the possibility of plaque contents being exposed to blood flow in case of rupture. In addition, the stent graft may provide reinforcement to the fibrous cap and reduce any physical stress placed on it due to the size of the lipid core.

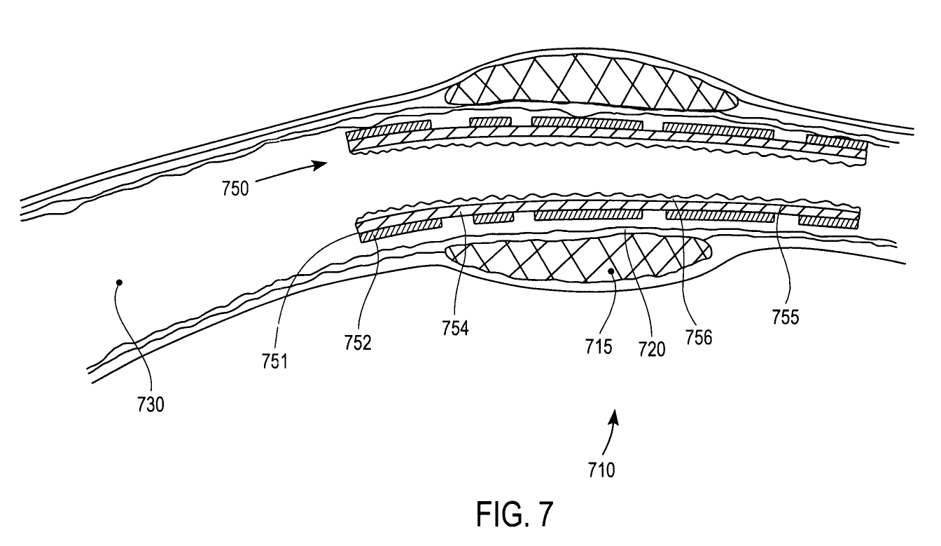

FIG. 7 illustrates another embodiment for treating vulnerable plaque in which stent graft 750 is deployed near vulnerable plaque 710. Stent graft 750 has a thin expandable polytetrafluoroethylene (ePTFE) cylindrical tube 754 affixed to inner surface 751 of self-expandable stent 752. Inner surface 755 of ePTFE tube 754 has a layer of endothelial cells 756. The layer of endothelial cells 756 promotes cell migration that eventually forms a complete monolayer on the surface of arterial lumen 730. As such, stent graft 750 shields existing vulnerable plaque 710 from an occlusive thrombosis event. Moreover, stent graft 750 may provide reinforcement to fibrous cap 720 and reduce any increased physical stress placed on it in vivo due to lipid core 715 presence or other hemodynamic forces.

ePTFE tube 754 serves as a physical barrier between vulnerable plaque 710 and arterial lumen 730. Because ePTFE lumen surface 755 acts as an arterial equivalent, ePTFE tube 754 should remain free from occlusion. In one embodiment, the ePTFE tube 754 is made anti-thrombotic by surface treatment. The surface of ePTFE tube 754 may be made anti-thrombotic for use as a vascular graft by seeding surface 755 with endothelial cells 756. Endothelial cells 756 seeded within vascular grafts have been shown to promote cell migration that eventually form a fully lined monolayer on a lumen surface.

Several approaches exist to seed stent graft 750 with endothelial cells 756. In one embodiment, a pressurized sodding technique may be used in which ePTFE tube 754 is expanded to 5 psi using media that contain endothelial cells. Endothelial cells 756 are isolated from the canine falciform ligament fat. Endothelial cells may also be isolated from human liposuction fat micro-vessel, umbilical veins, and other comparable sources.

Stent graft 750 may be disposed near a target vulnerable plaque 710 in a manner similar to that of a drug eluting stent 450, 550 at an occlusive site discussed above (e.g., with respect to FIGS. 4 and 5A-5C). Stent graft 750 is disposed near a distal end of a catheter (not shown). The catheter is passed through arterial lumen 730 so that stent graft 750 is positioned near vulnerable plaque 710. A retractable sheath (not shown) maintains the stent graft in a crimped position so that the stent graft may be advanced within arterial lumen 730. As illustrated in FIG. 7, stent graft 750 expands and applies physical pressure to fibrous cap 720 surrounding vulnerable plaque 710. FIG. 7 illustrates stent graft 750 in a fully expanded position, allowing normal blood flow through arterial lumen 730.

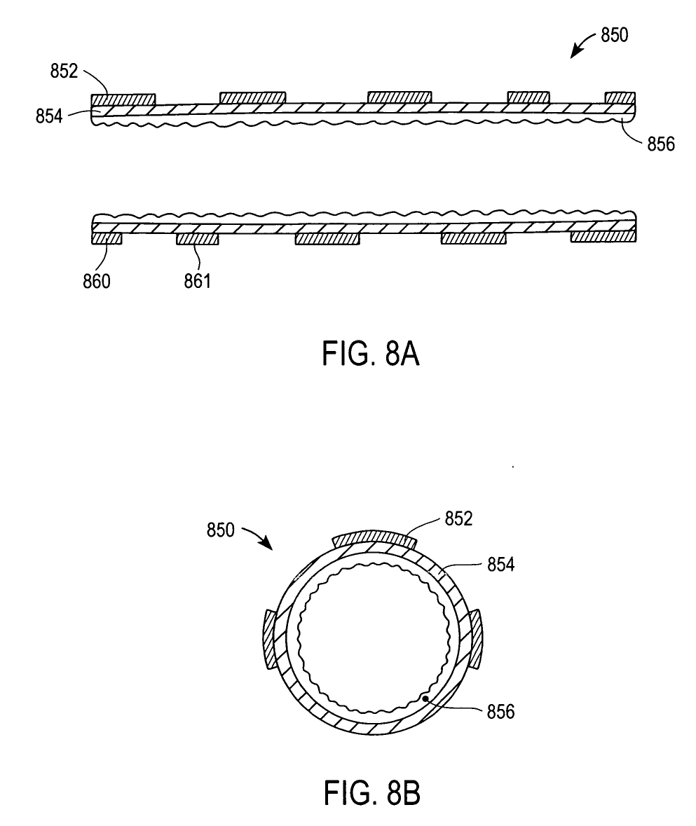

FIGS. 8A-8B illustrate cross-sectional views of stent graft 850 having inner tube 854 lined with endothelial cells 856 for treating vulnerable plaque. A self-expandable stent 852 is used as structural support to keep stent graft 850 secured in place within arterial lumen 730. A self-expandable stent may be advantageous over a balloon expandable stent. A self-expandable stent does not require an internal lumen pressure to expand, and so any seeded cells 856 are kept intact. In contrast, a balloon expandable stent could damage cells 856 of stent graft 850 when expanded. The self-expanding stent 852 may be made from a shape memory alloy such as NiTi (e.g., Nitinol). In order to provide additional flexibility to stent 852, stent links (e.g., 860, 861) may be eliminated from stent 852. In an alternative embodiment, stent 852 may have a series of shape memory metallic rings (not shown) bonded to the outer surface of ePTFE tubing 854.

Various techniques are available to bond ePTFE tube 854 to stent 852. For example, to bond the ePTFE tube to the metal, a primer is first applied to the metallic portions (e.g., 860, 861) of stent 852. These rings are then inserted over ePTFE tube 854. Silicon adhesive is used to bond metallic rings 860, 861 to ePTFE tubing 854. The stent graft is cured at about 160° C. for approximately 15 minutes. The silicon adhesive seeps through the ePTFE tube matrix and after curing acts as a medium that mechanically fastens the ePTFE tube to the metal. The inner surface of the polymeric tube is then seeded with endothelial cells.

In addition to the shape memory alloys, stent rings 860, 861 may also be made from shape memory polymers. Various shape memory polymers with great potential for biomedical applications are currently in the research phase. For example oligo(e-caprolactone)dimethacrylate and n-butyl acrylate are two monomeric compounds that, when combined, generate a family of polymers that exhibit excellent shape memory characteristics. The oligo(e-caprolactone)dimethacrylate furnishes the crystallizable “switching” segment (characteristic of shape memory materials) that determines both the temporary and permanent shape of the polymer. By varying the amount of the comonomer, n-butyl acrylate, in the polymer network, the cross-link density can be adjusted. This allows the mechanical strength and transition temperature of the polymers to be tailored over a wide range. Therefore, the stent incorporating these polymers can be deployed using their shape memory characteristics. Furthermore, other polymers such as polyurethane and ultra high molecular weight polyethylene (UHMWPE) can also be used for tubing used in the stent graft.

In an alternative embodiment, stent graft 850 may also be used as an apparatus for local drug delivery. Stent graft 850 may be loaded with anti-restenotic, anti-thrombotic, or other vulnerable plaque treatment agents (e.g., as discussed above with respect to FIGS. 4 and 5A-5C). Furthermore, in yet another alternative embodiment, stent graft 850 may be radioactively enhanced or incorporated with a material that generates a magnetic susceptibility artifact of stent graft 850.

Needle Catheter

In another embodiment, a vulnerable plaque treatment drug or biologically active agent may be injected through or around a vulnerable plaque region. In one embodiment, a needle catheter may be used to inject the biologically active agent. The needle catheter may be adjusted to penetrate various targets around the vulnerable plaque including, but not limited to: fibrous cap, proteoglycan-rich surface layer, subintimal lipid core, proximal or distal regions of the vulnerable plaque, media containing smooth muscle cells around the lipid core, and the periadventitial space.

In an alternative embodiment, the needle catheter may include sensing capabilities to determine the depth of penetration of the needle, as well as dial-in needle extension. Furthermore, different angle balloons may be added in order to use case-specific ramp angle to penetrate into the vulnerable plaque region while positioning the needle catheter below the actual occlusion. The needle catheter may be placed proximal or distal to the vulnerable plaque region because studies have shown cell localization, activity, and apoptosis have preferential occurrence in the upstream or downstream parts of vulnerable plaque regions.

FIG. 9A illustrates a cross-sectional view of one embodiment of needle catheter 950 that may be used to inject a vulnerable plaque treatment agent into arterial wall 980 near vulnerable plaque 910. Vulnerable plaque 910 has developed within arterial wall 980, separated from arterial lumen 930 by fibrous cap 920. Distal end 941 of catheter 940 has inflatable balloon 948 with at least one needle lumen 945 extending from distal end 941 of catheter 940 along proximal end 947 of balloon 948. Retractable needle 945 extends from needle lumen 942 and penetrates arterial wall 980. Inflated balloon 948 secures needle catheter 950 at a target location. Moreover, because needle sheath 942 is coupled along proximal end 947 of balloon 948, inflated balloon 948 provides a penetration angle for needle 945. Needle catheter 950, as illustrated, has two needles 945, 946 extending from distal end 941 of catheter 950. Any number of needles may be utilized with needle catheter 950. For example, in an alternative embodiment, the needle catheter may have only one needle for injecting a vulnerable plaque treatment agent.

As illustrated, needle catheter 950 targets lipid core 915 of vulnerable plaque 910 directly. In one embodiment, a lipid lowering agent may be injected into vulnerable plaque 910, or agents which could change lipid core properties could be injected. PEG with an aldehyde/gluteraldehyde mix, genipin, or a di or poly PEG-NHS ester such as PEG bis-Succinimidyl α methylbutanoate (Nektar), may be injected into lipid core 915 potentially cross-linking vulnerable plaque 910 components to inhibit erosion, rupture, or other forms of destabilization. Other vulnerable plaque treatment agents may be used, including antioxidants, and extracellular matrix synthesis promoters (e.g., as discussed with respect to FIGS. 4 and 5A-5C).

Needle catheter 950 may also be configured to include a feedback sensor (not shown) for mapping the penetration depth of needles 945, 946. The use of a feedback sensor provides the advantage of accurately targeting the injection location. Depending on the type of treatment agent used and treatment desired, the target location for delivering the treatment agent may vary. For example, it may be desirable to inject a drug near fibrous cap 920 or media 984 of arterial wall 980. Alternatively, it may be desirable to inject a drug into lipid core 915, or adventitia 986.

In use, distal end 941 of needle catheter 950 is inserted into the lumen of a patient and guided to a vulnerable plaque region. As illustrated in FIG. 9A, distal end 941 of needle catheter 950 is positioned near a proximal end 912 of vulnerable plaque 910. Alternatively, needle catheter 950 may be positioned near a distal end 914 of vulnerable plaque 910. Vulnerable plaque 910 may be detected using the sensor (not shown) disposed on needle catheter 950. By utilizing a sensor, the injection site for treating vulnerable plaque 910 may be precisely identified.

FIGS. 10A and 10B illustrate cross sectional views of one embodiment of a needle catheter for injecting a vulnerable plaque treatment drug or biological agent. FIG. 10 illustrates needle catheter 1001 with sensing capabilities having elongated catheter body 1010 that surrounds needle lumen 1012 and inner lumen 1014. Housed within inner lumen 1014 are fluid lumen 1016 and inner member 1018 that also contains guide wire 1020, guide wire lumen 1022, and ultrasonic element lumen 1024. Inflatable balloon 1026 is coupled to inner lumen 1014 and the inner member 1018. Proximal end 1028 of balloon 1026 is coupled to distal end 1030 of inner lumen 1014 and distal end 1032 of balloon 1026 is coupled to distal end 1036 of inner member 1018.

In an alternative embodiment, both guide wire 1022 and retractable ultrasonic element 1034 may be housed within inner member 1014. Elongate body 1010 surrounds inner member 1014 and needle lumen 1012. Housed within inner lumen 1014 are inner member 1018 and fluid lumen 1016. Inner member 1018 surrounds guide wire 1022 and retractable ultrasonic element 1034. Inflatable balloon 1026 is coupled to inner lumen 1014 and inner member 1018. Proximal end 1028 of balloon 1026 is coupled to distal end 1030 of inner lumen 1014 and distal end 1032 of balloon 1026 is coupled to distal end 1036 of inner member 1018.

The ultrasonic element lumen 1024 of inner member 1018 houses retractable ultrasonic element 1034. The distal end of the ultrasonic element has an ultrasound transducer or transducer array and the proximal end contains the associated co-axial cable that connects to an imaging display system (not shown). Ultrasonic waves generated by the ultrasonic element impinge on the surface of a vulnerable plaque or vulnerable plaque region. The timing/intensity of the ultrasonic waves reflected back to the transducer differentiates between the various anatomic boundaries or structures of the vulnerable plaque region, for example, the various layers of an arterial wall. The waves detected by the transducer are converted to electric signals that travel along the coaxial cable to the imaging system. The electrical signals are processed and eventually arranged as vectors based on the digitized data. In one embodiment, the ultrasound transducer has piezoelectric crystal configured for optimal acoustic output efficiency and energy conversion. In alternative embodiments, the crystal is made of PZT or lead-ceramic materials such as PbTiO₃ (lead titanate) or PbZrO₃ (lead zirconate).

As further illustrated in FIGS. 10A-10B, retractable needle 1013 is housed in needle lumen 1012 and freely movable therein. The hollow, tubular shaped needle 1013, having an inner diameter within a range of approximately 0.002 inch to 0.010 inch (5.1×10⁻³ cm to 25.4×10⁻³ cm) and an outer diameter within the range of approximately 0.004 inch to 0.012 inch (10.2×10⁻³ cm to 30.5×10⁻³ cm), provides a fluid channel that extends from proximal end 1040 to distal end 1042 of needle 1013. Distal end 1042 of needle 1013 has a curved tip. In one embodiment, needle 1013 has an angle of curvature of about 30 degrees to 90 degrees. The curvature of needle 1013 facilitates placement of the needle tip near or within a desired target of a vulnerable plaque region. Needle 1013 may be formed from a variety of metals including, but not limited to stainless steel, NiTi (nickel titanium) (e.g., Nitinol) or other comparable semi-rigid materials.

Proximal end 1040 of needle 1013 is coupled to adapter 1050 that couples needle 1013 to needle lock 1052 and needle adjustment knob 1054. Needle lock 1052 is used to secure needle 1013 in place and prevent further movement of needle 1013 within an arterial lumen once needle 1013 is placed in the target position. Needle adjustment knob 1054 controls accurate needle extension out of the distal end of the catheter and depth of penetration into the vulnerable plaque region. As such, movement of needle adjustment knob 1054 moves needle 1013 in and out of needle lumen 1012. Once needle 1013 has penetrated a target to a desired depth, needle lock 1052 enables needle 1013 to be secured in place thereby preventing any movement of needle 1013 within needle lumen 1012.

A drug injection port 1060 is disposed near proximal end 1062 of needle catheter 1001. Drug injection port 1060 couples needle catheter 1001 with various dispensing devices such as a syringe or fluid pump. Fluids injected into drug injection port 1060 travel through needle 1013 and are dispensed from the distal tip of needle 1013.

FIGS. 9B-9D illustrate embodiments of needle catheter 950 targeting various regions near a vulnerable plaque for injection of a vulnerable plaque treatment agent. As discussed above, needle catheter 950 may have a feedback sensor (e.g., ultrasonic element 1034 of FIG. 10B) to determine and control a penetration depth for needles 945, 946. The sensor provides the advantage of accurately targeting a desired injection site. As such, needle catheter 950 may inject a vulnerable plaque stabilizing drug or biologically active agent into fibrous cap 920 as illustrated in FIG. 9B, regions within the subintimal space 982 of arterial wall 980 as illustrated in FIG. 9C, or regions distal to vulnerable plaque 910 as illustrated in FIG. 9D.

For example, with respect to FIG. 9B antioxidants such as reactive oxygen scavengers (ROS), vitamin C and E may be injected into fibrous cap 920. The oxidant acts as a matrix-ase inhibitor to prevent significant collagen degradation within fibrous cap 920.

In another embodiment, needle catheter 950 may also be used as part of a biological or gene therapy method to treat vulnerable plaque 910. For example, upregulators of tissue inhibitors of metalloproteinases (TIMPS) may be injected into adventitia 986. TIMPS are expressed by surrounding smooth muscle cells to downregulate MMP production. Alternatively, recombinant TF pathway inhibitors (TFPI) may one day be injected into lipid core 915 to inhibit thrombosis due to erosion, rupture or other forms of plaque destabilization.

In yet another embodiment, needle catheter 950 may be used to deliver an agent to induce angiogenesis and/or arteriogenesis as described above with respect to FIG. 12. The therapeutic angiogenesis agents and drugs discussed above may be injected near a treatment site as an alternative to delivery by a drug eluting stent.

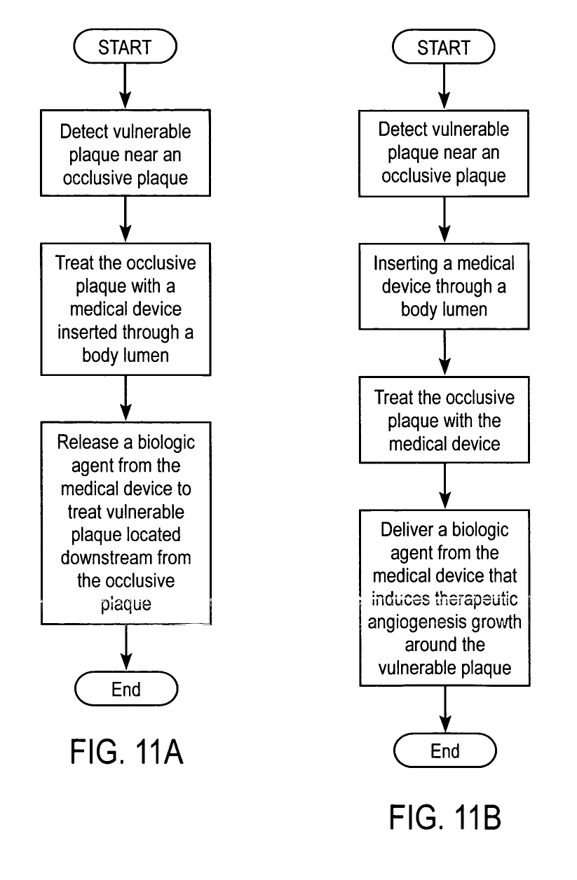

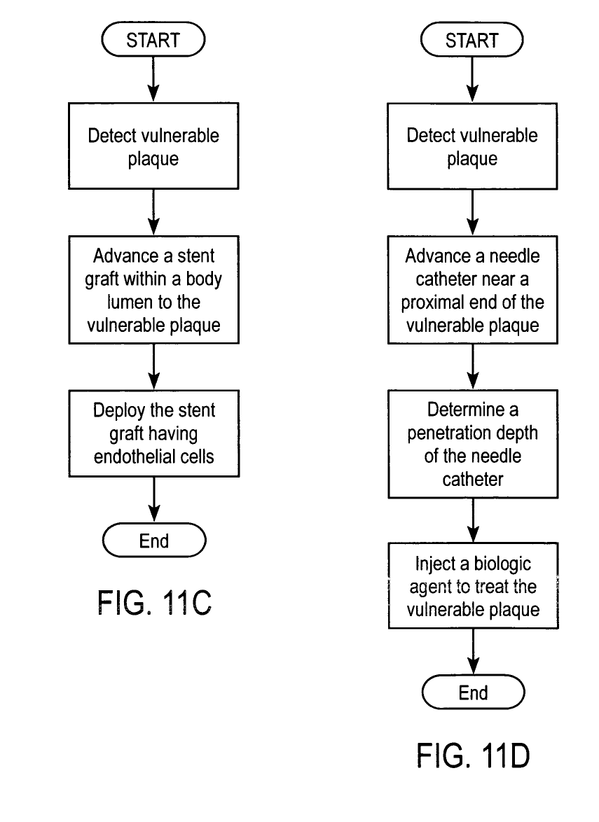

FIGS. 11A-11D illustrate flowcharts describing methods for stabilizing vulnerable plaque. The methods described with respect to FIGS. 11A-11D include detecting vulnerable plaque. Various techniques may be utilized to detect the presence and location of vulnerable plaque. For example, an ultrasound probe (IVUS) or an optical coherence tomography probe (OCT) may be guided through the arteries to scan for vulnerable plaque. Alternatively, magnetic resonance imaging (MRI) devices may be able to detect vulnerable plaque. Near Infrared spectroscopy is another technique for detecting vulnerable plaque. For example, certain wavelengths of light penetrate the arterial wall and produce a specific chemical signature that could correlate to vulnerable plaque composition. Additionally, thermography may also be used to detect vulnerable plaque. Plaques that rupture tend to be inflamed, and data indicates this correlates to a higher temperature compared to non-vulnerable type plaques that do not rupture. As such, a temperature sensitive probe that measures the temperature of arteries could indicate the presence of vulnerable plaque. Alternatively, liquid crystal thermography methods may also be used. For example, a balloon material made of a thermochromic liquid crystal material may be able to optically detect property changes when exposed to increases in temperature. When the balloon contacts a vulnerable plaque, the higher temperature of the vulnerable plaque may be detected by analyzing a beam of light directed towards the suspected vulnerable plaque region and the balloon material in contact therewith. The light may undergo a color change in the balloon material as a result of the higher temperature.

FIG. 11A describes a method to treat vulnerable plaque downstream from an occlusive plaque. The occlusive plaque may be treated with a stent or balloon catheter. The vulnerable plaque may be treated by altering the lipid core and/or strengthening or thickening the fibrous cap surrounding the vulnerable plaque. The vulnerable plaque is first detected by any one of the techniques described above, including but not limited to IVUS, OCT, MRI, near infrared spectroscopy, thermography, and liquid crystal thermography. The vulnerable plaque may be downstream from an occlusive plaque that has been detected, for example, with an angiogram. A drug delivery catheter is provided having a vulnerable plaque stabilizing agent. In one embodiment, the drug delivery catheter may deploy a drug eluting stent. The drug eluting stent is positioned at the occlusive plaque to widen the arterial lumen whose blood flow has been impeded by the plaque. The vulnerable plaque stabilizing agent is released towards a vulnerable plaque region located downstream from the release site. Alternatively, the agents may be in the form of microparticles to control the release of the agents over time. The agents released from the drug delivery catheter may include lipid lowering agents, antioxidants, extracellular matrix synthesis promoters, or inhibitors of plaque inflammation and extracellular degradation.

FIG. 11B describes a method to treat vulnerable plaque by inducing collateral artery or vessel growth to the myocardium downstream from or adjacent to an occlusive plaque. The occlusive plaque may be treated with a stent or balloon catheter. By inducing therapeutic angiogenesis (e.g., collateral artery or vessel growth), blood flow is maintained in case a vulnerable plaque ruptures leading to an occlusive thrombosis. The vulnerable plaque is first detected by any one of the techniques described above, including but not limited to IVUS, OCT, MRI, near infrared spectroscopy, thermography, and liquid crystal thermography. The vulnerable plaque may be downstream from an occlusive plaque that has been detected, for example, with an angiogram.

A drug delivery catheter or stent is provided having an agent that induces collateral artery or vessel growth. In one embodiment, the drug delivery catheter may deploy a drug eluting stent. The drug eluting stent is positioned at the occlusive plaque to widen the arterial lumen whose blood flow has been impeded by the plaque. The agent to induce collateral artery or vessel growth is released towards a vulnerable plaque region located downstream from the drug release site. Representative therapeutic or biologically active agents include, but are not limited to, proteins such as vascular endothelial growth factor (VEGF) in any of its multiple isoforms, fibroblast growth factors, monocyte chemoatractant protein 1 (MCP-1), transforming growth factor alpha (TGF-alpha), transforming growth factor beta (TGF-beta) in any of its multiple isoforms, DEL-1, insulin like growth factors (IGF), placental growth factor (PLGF), hepatocyte growth factor (HGF), prostaglandin E1 (PG-E1), prostaglandin E2 (PG-E2), tumor necrosis factor alpha (TBF-alpha), granulocyte stimulating growth factor (G-CSF), granulocyte macrophage colony-stimulating growth factor (GM-CSF), angiogenin, follistatin, and proliferin, genes encoding these proteins, cells transfected with these genes, pro-angiogenic peptides such as PR39 and PR11, and pro-angiogenic small molecules such as nicotine.

FIG. 11C describes a method to treat vulnerable plaque by implanting a stent graft on the arterial wall near a vulnerable plaque. This method of vulnerable plaque stabilization may be performed independent of treating an occlusive plaque. The vulnerable plaque is first detected by any one of the techniques described above, including but not limited to IVUS, OCT, MRI, near infrared spectroscopy, thermography, and liquid crystal thermography. The stent graft is disposed near a distal end of a catheter and advanced within the arterial lumen and positioned near a vulnerable plaque. Retracting a sheath covering the stent graft deploys the stent graft. In one embodiment, the stent graft has a thin ePTFE cylindrical tube affixed to the inner surface of a self-expandable stent. The inner surface of the stent has a layer of endothelial cells. The layer of endothelial cells promote cell migration that forms a fully lined monolayer on the arterial lumen surface. As such, the stent graft shields existing vulnerable plaque from an occlusive thrombotic event. Moreover, the stent graft provides reinforcement to the fibrous cap and reduces any physical stress placed on it due to the presence of the lipid core and hemodynamic forces.

FIG. 11D describes another method to treat vulnerable plaque. The vulnerable plaque may be treated by injecting a stabilizing drug or biologically active agent at various locations within and around the vulnerable plaque. The vulnerable plaque is first detected by any one of the techniques described above, including but not limited to IVUS, OCT, MRI, near infrared spectroscopy, thermography, and liquid crystal thermography. A needle catheter is advanced through an arterial lumen and positioned near a proximal end of the vulnerable plaque. Alternatively, the needle catheter may be positioned at or near a distal end of the vulnerable plaque. A sensor disposed on the needle catheter determines a penetration depth for the needle catheter. The needle catheter may be adjusted to penetrate various targets around the vulnerable plaque including, but not limited to: fibrous cap, proteoglycan-rich surface layer, subintimal lipid core, proximal or distal regions of the vulnerable plaque, media containing smooth muscle cells above the lipid core and the adventitial space. The agents released from the drug delivery catheter may include lipid lowering agents, antioxidants, extracellular matrix synthesis promoters, inhibitors of plaque inflammation and extracellular degradation.

Other methods involving a combination of devices and/or drugs may also be used to treat a diseased artery containing discrete lesions, diffuse disease, or vulnerable plaque. Combination therapy allows different drugs to be administered into the artery by different means. Combination therapy provides an advantage of delivering one or more drugs via different paths to augment or synergize the effect of another. For example, one method uses an implantable drug eluting device like a drug eluting stent or a stent graft, coated with a first drug for direct absorption by the arterial walls, in combination with retrograde perfusion, where a second drug is infused into a venous vessel through retrograde pressurized perfusion towards the arterial vasculature. The artery can benefit from one mode of absorption of one drug from a drug eluting stent at the lesion site and another mode of absorption of a second drug delivered to surrounding myocardial vasculature through diffusion by way of retrograde pressurized infusion. A combination therapy is aimed to augment or complement the effect of one drug, delivered in one manner such as an eluting stent, with a second or third drug, delivered in a different manner such as retrograde perfusion, to enhance the overall therapeutic treatment of multiple drug delivery to the diseased vessel. The following embodiments describe different methods of drug delivery by combination therapy.

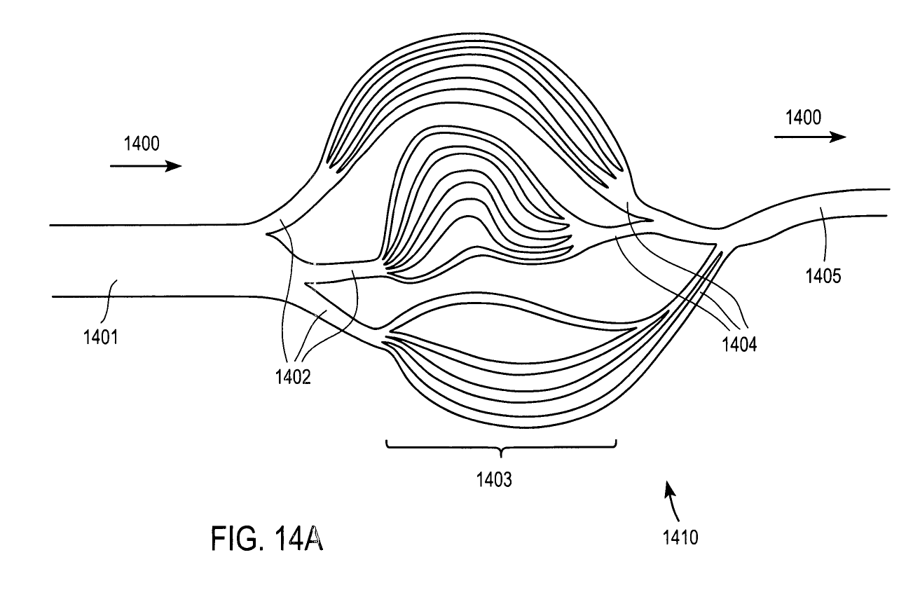

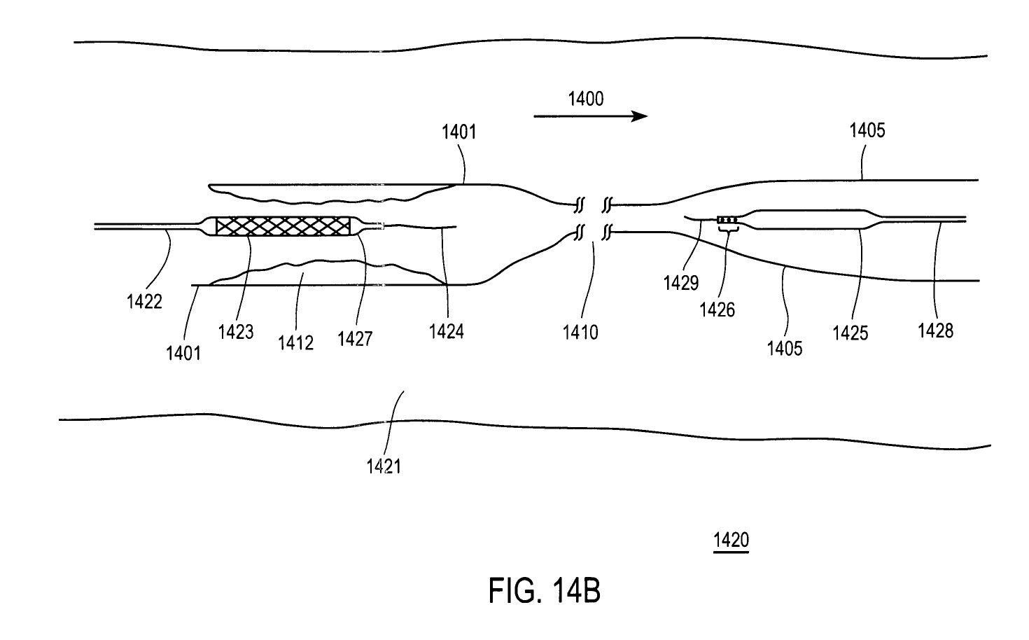

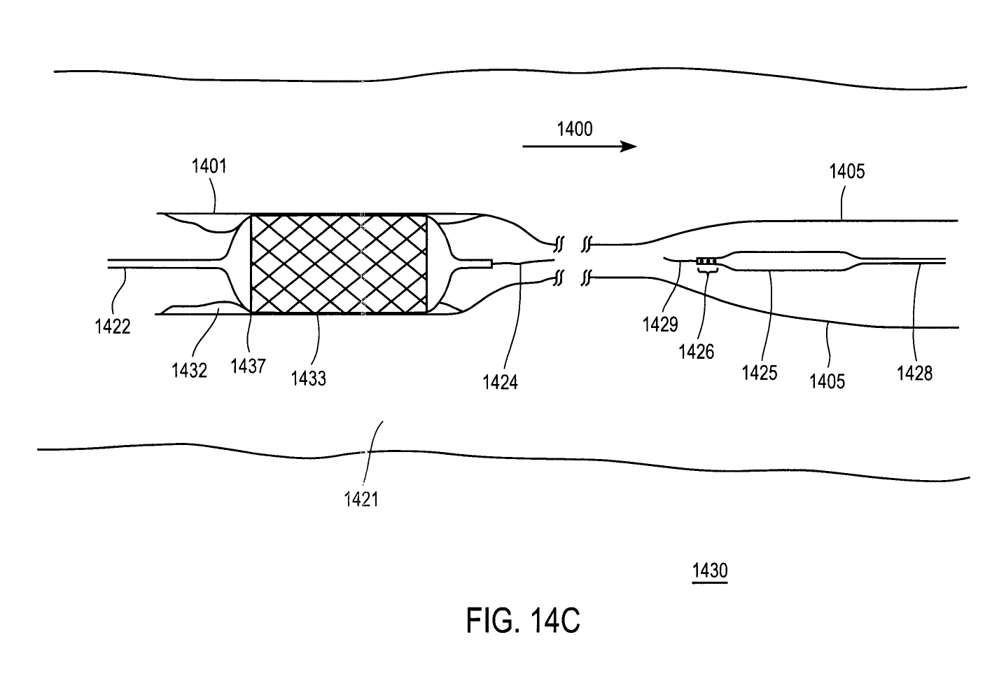

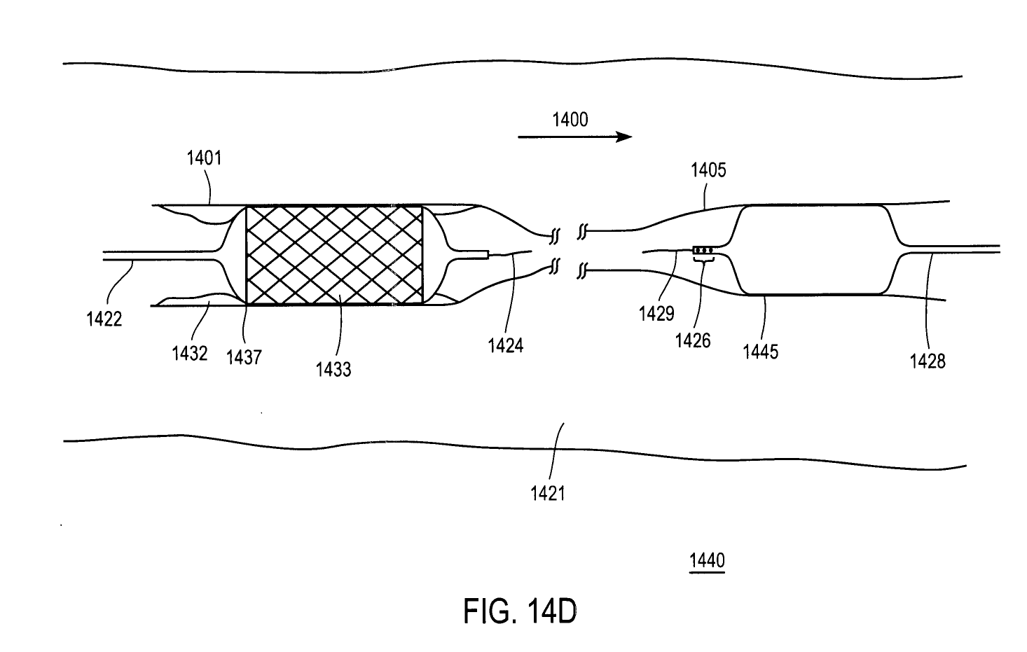

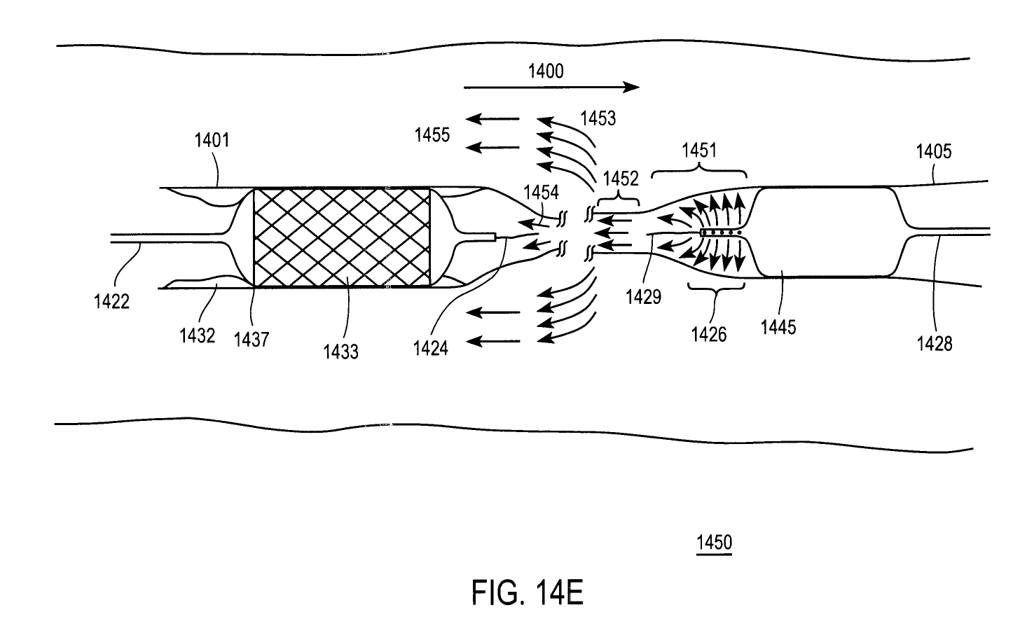

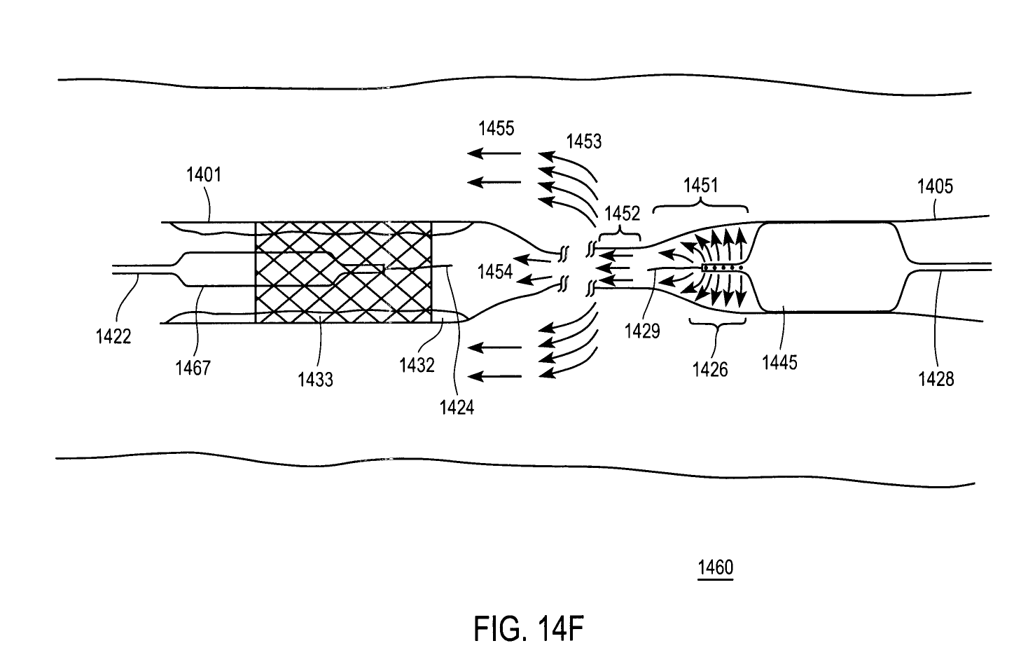

FIG. 14A to 14F describe the combination therapy of administering one drug via a drug eluting stent and a second drug via a venous vessel by way of retrograde perfusion. FIG. 14A illustrates the microscopic tissue vasculature where oxygenation and nutrient exchange takes place. Oxygenated blood flows from the artery 1401 in the direction 1400 into arterioles 1402 which then flows into a network of capillaries 1403. After the oxygen and nutrient exchange takes place in the capillaries, blood flows and circulates into the venules 1404 then through the veins 1405 and return to the heart.

Nutrients, waste products and oxygen exchange takes place within a network of capillaries via diffusion as a result of the differences in concentration of nutrients, waste products and oxygen between the blood in the capillaries and the surrounding tissue. As blood runs from the artery into the arterioles through the capillaries into the venules and then the veins, the blood pressure drops. The pressure is much larger on the arterial side 1401 (e.g., 60-160 mmHg), with the bulk of the pressure dropped at the arterioles 1402 by their muscular constriction, such that the pressure at the level of the capillaries 1403 and beyond into the venous system (1404 and 1405) is normally very low (e.g., 5-15 mmHg). In a situation where the tissues surrounding the capillaries fed by an arteriole become ischemic, the arteriole will open more and allow more blood pressure or flow to be applied to those capillaries. Gas exchange takes place in the capillaries, via diffusion driven by a difference in concentration, called a concentration gradient. For instance, oxygen is more abundant (at a higher concentration) inside the arterial blood flowing into the capillaries as compared to the surrounding tissue, thus, as a consequent of this concentration gradient, oxygen will diffuse from a high concentration area, such as blood in the capillaries, into a low concentration area, such as the tissue, through the capillary walls. On the contrary, carbon dioxide, a waste product, is more abundant in the surrounding tissue than the blood inside the capillaries, therefore, carbon dioxide diffuses from a higher concentration region of the tissue into the lower concentration region of the blood across the capillaries. In a very real sense, a difference in concentration of a substance in solution on either side of a permeable membrane (tissue cell walls), like that may exist between the blood and the internal portions of the cells in a tissue, can be viewed as pressure differential, in that, to stop the diffusion of the substance from an area of greater concentration across the membrane to an area of lesser concentration, a pressure must be applied across the membrane with the higher pressure on the side of the membrane with the lesser concentration. The pressure required to stop the diffusion of the substance due to the concentration difference can be considered the pressure of diffusion of the substance at that concentration difference. Conversely, the application of a high enough pressure (greater than the pressure of diffusion) on one side of a membrane can drive a substance through the membrane/into tissues, regardless of the relative concentrations of the substance on either side of the membrane. Ion exchange also takes place via the cell membrane of the capillaries via diffusion and/or active transport. Other larger nutrients, if capable of diffusing from the capillaries into the surrounding tissue driven by a difference in concentration (or concentration gradient), are often hydrophobic in nature. This is because cell membranes are made of lipoprotein, which is hydrophobic in nature, and all materials that are also hydrophobic or lipid soluble can easily diffuse across the semi-permeable membrane driven by a concentration gradient via passive transport. Otherwise, the larger nutrients are carried across the cell membrane via an active transport mechanism.

Understanding the mechanism and manner in which molecular transport or exchange takes place at the capillaries is important to select the proper drug and identify the most effective means of performing retrograde perfusion. Retrograde perfusion is a method of delivering molecules into the tissue or surrounding arterial vasculature against the normal flow of blood, from the veins in the direction of the arteries through the capillaries. FIGS. 14B through 14F describes the process of administering a combination therapy of using a drug eluting stent along with delivering a drug in retrograde from the veins with the use of a drug delivery catheter.

FIG. 14B shows a stent delivery catheter 1422 with the aid of a guidewire 1424 delivering a collapsed drug eluting stent 1423 mounted over an unexpanded balloon 1427 into a diseased artery 1401 with a lesion 1412 such as a discrete blockage, diffuse disease or vulnerable plaque. Distal to the artery is a vascular network of arterioles, capillaries and venules 1410 (not shown in the figure) within the myocardial tissue 1421. On the other end of the vascular network, a drug delivery catheter 1428 with an unexpanded balloon 1425 and ports 1426 at the distal end of the drug delivery catheter 1428 is delivered into a vein 1405 distal to the vascular network of capillaries and venules 1410 relative to the direction of blood flow 1400 with the aid of a guide wire 1429. Note that the guide wires 1424 and 1429 used for arterial and venous deliveries of the different devices in FIGS. 14B through 14F can be the same or different based upon the user's preference, the characteristics of the anatomy and vasculature, and the devices in which it is delivering. They can be identical or different in any one or more characteristics such as length, diameter, and tip stiffness etc.

FIG. 14C shows the inflation of balloon 1437 and expansion of the drug eluting stent 1433 to treat the lesion 1432 in the artery while the drug delivery catheter 1428 remains in place and un-inflated as in FIG. 14B. The drug eluting stent can be deployed to treat the lesion directly, proximal or distal to the lesion. Placement of the drug eluting stent depends on the objective of the user physician. The choice of stent and location of placement depends on whether the stent will disrupt or stabilize the lesion if it is a vulnerable plaque or whether the drug eluting stent is used to treat an occlusive atherosclerotic plaque which is upstream of a vulnerable plaque whereby the drug or biologically active agent released from the stent is intended to treat the vulnerable plaque region downstream. The method in coating a stent and drug selection for a stent are described earlier in this application and are equally applicable in this case. FIG. 14D shows that an expanded balloon 1445 on the drug delivery catheter is inflated to occlude blood flow in preparation for drug injection into the venous space between the balloon and the capillaries for retrograde perfusion. Since the veins typically have much thinner and much more elastic walls than the arteries, the balloon on the drug delivery catheter is likely to be made of a more compliant or elastic materials such as low density polyethylenes, Pebax®, silicone and the like as opposed to cross-linked Nylons, high density polyethylenes, polyethylene terephthalate (PET) and the like which are more often used for expansion of a stent in a narrow range of diameters, and to be deployed at a much lower pressure compared to stent delivery, in order to minimize trauma and injury to the veins and still provide a wide range of occlusive diameters to the highly elastic veins.

FIG. 14E shows that while the drug eluting stent 1433 is in place with the stent delivery balloon 1437 expanded and the compliant balloon 1445 is expanded on the drug delivery catheter to occlude blood flow, a drug is injected into the venous lumen 1451 via the port holes 1426. As the drug travels in reverse or retrograde 1452 through the vein and the venules into the capillaries, the pressure in the venules and the capillaries will rise and the drug will be pushed 1453 through the capillary walls into the tissue and further disperse 1455 deeper into the tissue. The dispersion of the drug will be positively driven into the tissue by both the pressure gradient of the increased pressure inside the capillaries and the concentration gradient of the drug. Some small portion of the drug may even travel in reverse back to the artery 1454 through the capillaries and the arterioles. FIG. 14F shows the continue injection of drug through the port holes of the drug delivery catheter in retrograde while the stent delivery balloon 1467 is deflated and with the stent 1433 remain expanded. The pressure to infuse drug through the delivery catheter may increase after the stent delivery balloon is collapsed, as the drug being delivered in retrograde will have to go against more pressure as blood pressure is now increased distal to the balloon in the artery previously occluded by the inflated stent delivery balloon.

The timing to inflate the balloon of the drug delivery catheter may be synchronized with the inflation of the balloon on the stent delivery catheter to deliver the drug eluting stent. For example, when the stent delivery balloon is inflated, the volume of blood flow distally into the venous system will reduce and so will the pressure. According to one embodiment, after the stent delivery balloon is inflated, the balloon of the drug delivery catheter is inflated and the drug delivered because there will be less pressure to resist the infusion of the drug. On the other hand, if the maximum benefit of the drug delivered by retrograde perfusion requires the drug on the stent to be absorbed into the arterial wall first, a much longer latency period may be required and the retrograde perfusion procedure may be performed on a separate occasion. Similarly, the reverse is true if a latency period is required between delivering the drug eluting stent after retrograde drug perfusion where the maximum synergistic effect is only achieved after the first drug delivered by the retrograde perfusion is completely absorbed into the tissue before the second drug is to be delivered to the lesion site by a drug eluting stent.

As explained earlier, the pressure used to inject the drug and the affinity of the drug is important to the retrograde perfusion drug delivery. On the one hand, the drug should complement or augment the action of the drug released by the drug eluting stent. For example, if the drug is an anti-inflammatory, anti-restenotic, anti-proliferative, or plaque stabilizing or treatment drugs such as lipid lowering agents, antioxidants, extracellular matrix synthesis promoters, inhibitors of plaque inflammation and extracellular degradation or estradiol drug classes and derivatives as described earlier in this application, the drug used for retrograde perfusion can be a therapeutic or biologically active agent used to induce therapeutic angiogenesis. The ideal drug selected will not only augment and complement the effect of the drug released by the drug eluting stent, it should be hydrophobic and small to promote tissue perfusion at the capillary interface. Furthermore, the pressure selected for drug injection should be larger than the arterial pressure exerted on the other end of the capillary bed so that the drug solution can be pushed as far back into the arterial side as is as possible.

The pressure and rate of drug injection should be constantly monitored to prevent rupturing the vein, venules or capillaries. This can be achieved by having a barometer sensor in the tip of the drug delivery catheter, monitoring the pressure via a separate catheter lumen or utilizing a pressure sensing guidewire. In addition, retrograde drug injection may encounter less resisting pressure if the stent delivery balloon remains inflated to reduce the arterial blood pressure applied to the capillaries, but this should not persist for a long period of time as it may cause ischemia in the myocardial tissue.

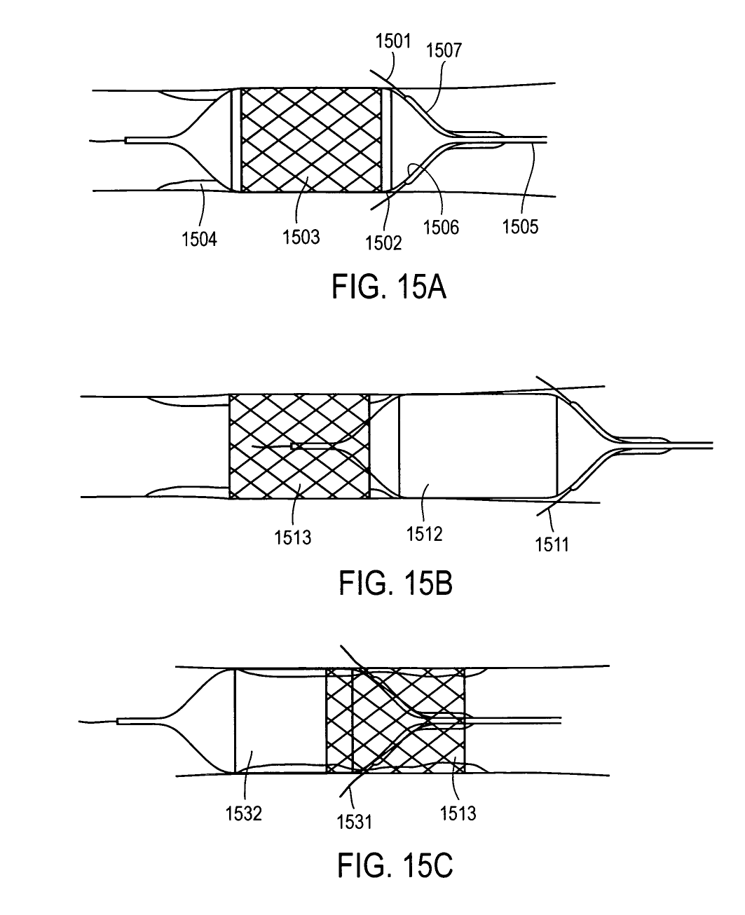

FIGS. 15A through 15C show yet another embodiment of a combination therapy carried out using a drug eluting stent with a needle catheter which also acts as a drug delivery system. In this embodiment, the needle catheter includes characteristics described earlier such as sensing capabilities to determine the depth of penetration of the needle, dial-in needle extension, and different shoulder angles on the balloon for different entry angles to treat different lesion types. The needle catheter also acts as a stent delivery system to deliver and deploy the drug eluting stent at the lesion site. After the balloon is inflated to expand the drug eluting stent in the lesion site, the needle is extended to penetrate the arterial wall for drug infusion. As described earlier, each needle catheter will have at least one injecting needle, although there may be up to four needles or more and each needle may be controlled and operated independently of each other.

The needle in the catheter shown in FIGS. 15A through 15C is hollow and is made of a shape memory alloy or polymer. Preferably, it is made of Nickel Titanium (NiTinol) where the needle can be both flexible and retains its shape after flexing. The needle may also be pre-shaped to take on a particular curve after advancing outside of the protective sheath in order to penetrate the vessel wall at an angle different than a typical straight needle. Ordinarily, a straight needle will only penetrate the vessel wall at an angle similar to that made by the balloon shoulder against the vessel wall. The needle control at the proximal handle of the stent delivery system controls the rotation of the needle, the depth penetration of the needle as well as the rate of drug infusion depending on the pressure applied. The controls for each of these parameters are preferably independent of each other.

FIG. 15A shows a needle catheter system 1505 acting as a stent delivery system to deliver and deploy a stent 1503 to treat a lesion 1504 in a diseased artery. After the stent 1503 is deployed and the balloon 1502 is inflated, the needle 1501 is advanced outside of a protective sheath 1507 along the shoulder 1506 of the balloon and into the tissue wall. Depending on the type of disease and lesion being treated, the needle insertion point should be approximately proximal to the lesion as indicated in FIG. 15A. As describe earlier, penetrating the lesion proximally may allow the needle to reach the lipid core interior and inject a lipid lowering agent to alter lipid core properties by increasing plaque stabilization and decreasing the likelihood of plaque rupture. Furthermore, the needle may be advanced to various depths. While the artery wall is divided into multiple layers including the internal elastic lamina, the media, the external elastic lamina and the adventitia, drugs can be injected into the layer where the target cells are located and where the different agents are most therapeutically effective. For example, to stimulate endothelial regrowth, agents are more effective injected in a depth just beyond the internal elastic lamina, while agents targeting reduction of fibrotic growth or anti-restenotic in nature are generally more effective in the media and the adventitia.

Similar in concept as FIG. 15A, FIGS. 15B and 15C show that the balloon 1502 of the needle catheter can be deflated and repositioned along the diseased artery to different locations and reinflated to inject the drug at different sites. FIG. 15B shows the balloon 1512 inflated proximal to the deployed stent and the needles 1511 penetrating the arterial wall at different depths for drug injection. Similarly, FIG. 15C shows the balloon 1532 inflated distal to the stent and the needles 1531 penetrating the arterial wall where the stent is placed for drug injection. Similarly the balloon may be deflated, re-positioned and re-inflated anywhere along a vessel such that the needles 1531 may penetrate the arterial wall distal to treat different regions.

Depending on the type of drug that is coated for release on the drug eluting stent, the treatment agent for injection into the arterial wall can be matched to complement or augment the drug effect of the drug eluting stent. Frequently, the therapeutic treatment desired will dictate the type of agent used, and the choice of agent is selected based upon the specific target lesion site. For example, while treating a diseased artery with both a vulnerable plaque and a discrete lesion, if the drug eluting stent has an anti-restenotic agent to treat the discrete lesion and to prevent future restenosis, another agent can be injected into the lipid core or the adventitia to complement the first drug by stabilizing the plaque to prevent erosion.

Similar to other embodiments previously described, the sequence and timing between administrations of the different therapies is dependent on the location and the maximum benefit derived from interaction of the drugs. Some drugs are best injected almost simultaneously to achieve a maximum benefit derived from the interaction of the drugs. Other drugs may be best injected with a latent period in between administrations because the maximum benefit may only be derived after a first drug has taken effect before a second drug should be applied. Therefore the timing and sequence in application of any combination therapy can be important and may depend on the drug(s) selected and the location in which they are injected.

Yet another embodiment of administering a combination therapy using the concept of a drug eluting stent and an injected agent is through the use of a modified hollow guide wire with a retractable drug injection needle in combination with a uniquely shaped balloon in a drug eluting stent system. This uniquely shaped balloon is specially extruded and then blown or molded where there is a trench or a trough giving the hollow guidewire access to the arterial wall for drug injection. Specifically, a stent or drug eluting stent mounted on this uniquely shaped balloon will not ride on the guide wire for access as a traditional balloon that has a wire lumen in the center of the balloon. Rather, the guide wire will ride in a guide wire lumen in the proximal member attached to the balloon shaft, over the groove or trench on the balloon and then back into a distal member attached to the balloon shaft distal to the balloon before exiting the catheter, to guide the catheter system to a lesion.

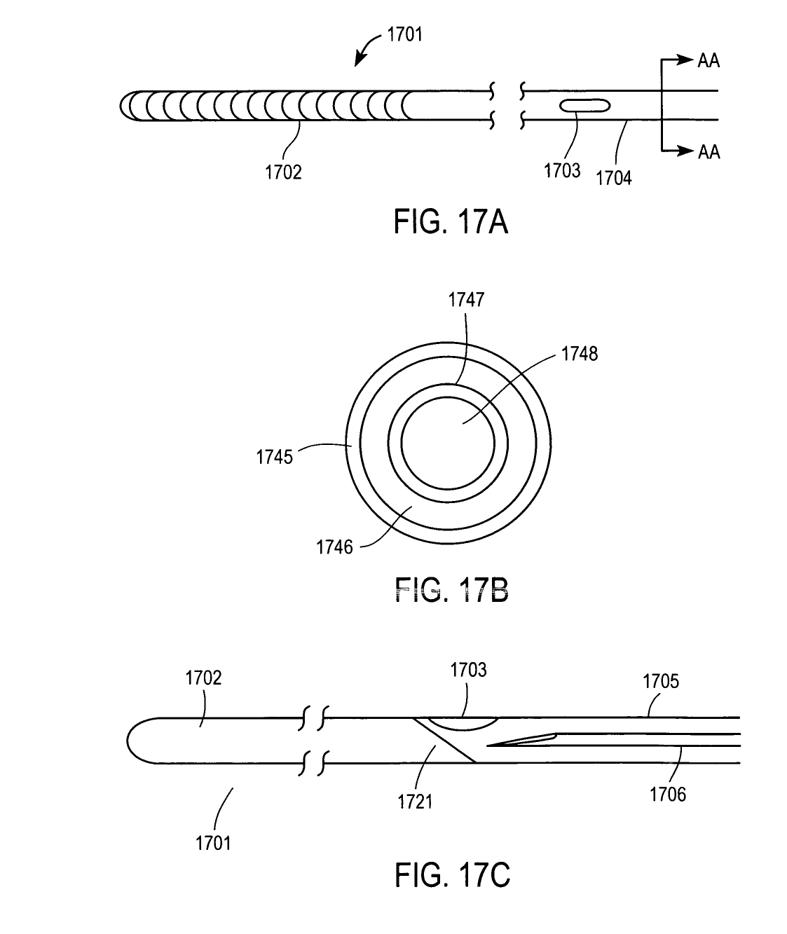

FIGS. 17A, 17B and 17C illustrate the structure of the modified guide wire with a needle. FIG. 17A shows a modified hollow guide wire 1701 with a typical floppy tip 1702 and a hollow hypotube body 1704. There is a port opening 1703 in the hollow hypotube body 1704 proximal to the distal section of the floppy tip 1702 for a hollow, retractable and flexible needle to advance outside of the hollow guide wire for drug infusion. The hollow hypotube body 1704 can be made of a metal like 316L stainless steel, metal alloy or polymer as used in a regular guide wire except with a hollow inner diameter large enough to house a needle. Similarly, the outer diameter and overall length of the modified guide wire should be comparable to most guide wires used for interventional procedures, including but not limited to the sizes currently available in the market such as 0.012″, 0.014″, 0.018″ and 0.035″. The length will also include but not limited to 180 cm for rapid exchange or 300 cm for over-the-wire applications in interventional procedures. The flexibility and the floppy tip stiffness of this hollow guide wire can be within a range as exhibited in various types of guide wires used for different applications in interventional procedures. For example, a floppy tip is generally used for atraumatic access to distal coronary anatomy, or a stiff tip which is for crossing a lesion. In any case, the flexibility and tip stiffness is not limited by the hollow structure of this new design. Similarly, the radiopacity in which the wire is visible under fluoroscope is not expected to be significantly limited because of the hollow wire shaft. In addition, the stiffness of the body of the guide wire is comparable with that of commercially available guide wire. The hollow guide wire itself may be made slightly more compliant and less stiff than a typical solid core guide wire; on the other hand, the modified guide wire may be slightly stiffer with the needle inserted inside the lumen of the modified guide wire.

FIG. 17B illustrates a cross-sectional portion AA-AA of the hollow hypotube as illustrated in FIG. 17A. The structure includes a hypotube, or guide wire cord forming the guide wire side wall 1745, surrounding a guide wire lumen 1746. Inside the guide wire lumen 1746 is a hollow, retractable, flexible, and sometimes pre-shaped needle with a needle side wall 1747. The needle lumen 1748 is for drug injection. Furthermore, the guide wire lumen 1746 may also be used to deliver drug for a range of different purposes such as delivering a muscle relaxant when the vessel becomes spastic while the balloon is inflated, or simply delivering a drug to bathe the vessel for therapeutic treatment. The sometimes pre-shaped needle is flexible and can be made of a super-elastic or shape memory metal alloy, such as Nickel Titanium (NiTinol), or a polymer. The gauge size or diameter of the needle will vary but range approximately between about 0.005″ to about 0.012″ in diameter for a typical 0.014″ coronary guide wire, the stiffness of the needle and/or the desired flow rate of drug in the needle during drug delivery. For example, in typical coronary applications, the outer diameter of the needle may preferably range between about 0.006″ to about 0.010″ while the guide wire may have an inner diameter ranging from approximately about 0.008″ to about 0.012″. Further, guide wire and injection needle wall thickness may range from about 0.001″ to about 0.003″ . Nevertheless, if applying this concept to about 0.012″ or about 0.035″ guide wires, the needle outer diameter may range from about 0.004″ up to about 0.032″. The overall flexibility of the modified guide wire 1701 with the flexible needle is comparable and within a range of similar guide wires with the corresponding diameters available in the market. For example, a guide wire floppy tip can be made of a core member having a proximal core section and a distal core section and a helical coil. The helical coil is disposed about and secured to the core member at its distal end by welding or soldering. This is but one of many alternate methods and ways to construct a guide wire tip. Similar to the guide wires available in the market, the tip of the modified guide wire may have different shapes and different degrees of stiffness for different applications. For example, the tip may be stiffer for crossing a total occlusion or it may be pre-shaped like a “J” while extremely soft for access to distal tortuous coronary vasculature while minimizing vessel trauma or spasms by reducing the wire's irritation to those sensitive regions.

FIG. 17C shows a longitudinal cross-sectional view of one embodiment of the modified hollow guide wire 1701. In this embodiment, the figure shows a flexible needle 1706 for drug injection near its resting position inside the hypotube 1705 near the port opening 1703. The distal tip 1701 of the guide wire, as described, is similarly constructed as traditional guide wires. The main body shaft of the guide wire proximal to the guide wire tip will be hollow with an exit port 1703 to allow for advancement of the drug delivery needle to exit the guide wire in penetrating the vessel wall for drug injection. This exit port is positioned at a specific range of distance from the tip of the guide wire. The distance is generally pre-determined to prevent having an excess length of guide wire distal to the tip of the catheter during drug injection or a lack of wire support because the exit port is too close to the floppy tip of the guide wire. For instance, the exit port 1703 on the guide wire should be approximately at least about 3 cm away from the transition of the distal floppy section of the guide wire assuming the shortest balloon of 10 mm is used. This distance is generally longer for longer balloons. Generally, the exit port should be placed in a location proximal to the transition of the floppy tip and wire body, longer than the sum of the catheter tip length, the distal taper length of the balloon and half of the balloon working length. Immediately distal to the exit port opening inside the guide wire lumen is a beveled solid stop or ramp 1721 at an angle directing towards the exit port to assist advancing the needle out of the exit port. If the needle is pre-shaped, the needle will take its natural pre-formed shape as it exits the port opening 1703. However, if the needle is not pre-shaped, the ramp 1721 can assist the needle in reaching a desired angle, similar to that of the ramp, in exiting the port opening and thus control the penetration angle of the needle into tissue. Further, the 1721 ramp can also act as a source of support in case the needle hits a plaque ridden lesion area. Thus, the ramp in conjunction with a pre-shaped guide wire can provide added support during needle penetration into the tissue.

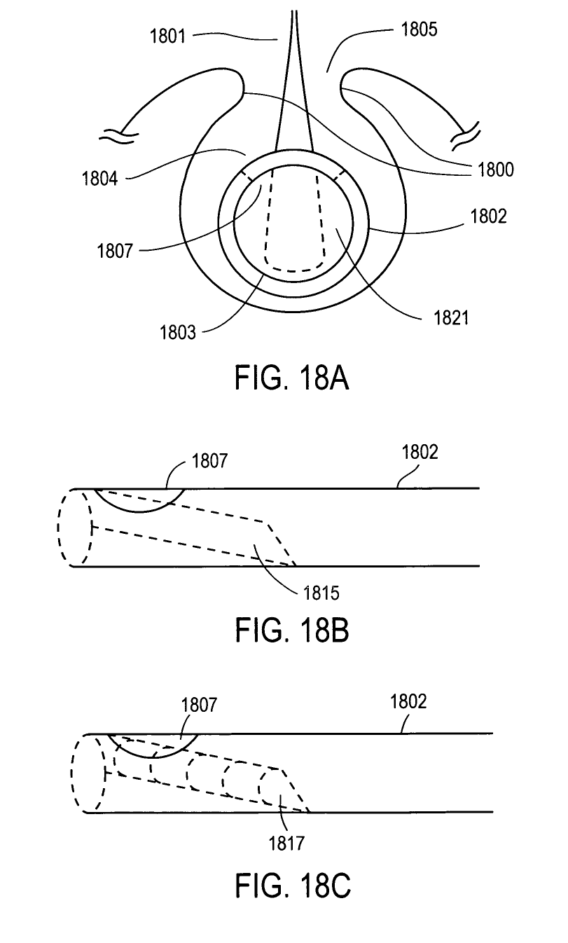

As would be understood by one skilled in the art, it is necessary to prevent puncturing of the balloon since the needle is to advance outside of the guide wire and into the vessel wall in the space provided by the balloon groove between side walls of the balloon. In one embodiment, at least two features can be used to prevent needle from puncturing the balloon. One feature is to use the solid stop or ramp to guide the drug injection needle while being advanced outside of the exit port opening from the guide wire. The direction and angle of the ramp are important in this aspect. FIG. 18A illustrates a needle 1801 extended from the needle exit port opening 1807 from the guide wire 1802. There is a ramp 1821 which angles from the base of the guide wire 1803 to the top of the guide wire 1804 where the port exit opening is located. The ramp is angled forward and up so that the needle can exit the port opening and point straight up in between the balloon walls 1800 into the space 1805 of the balloon groove opening. On the contrary, if the ramp is angled sideways, the needle will likely exit the port opening angling towards part of the balloon and its side wall and thus puncturing the balloon in the process. FIGS. 18B and 18C show two different configurations of the ramp 1821. FIG. 18B shows a ramp that is a flat bevel angling forward towards the tip of the guide wire and up towards the port opening. This is similar to the ramp as described in FIG. 18A. FIG. 18C shows a slightly different ramp. Instead of a flat bevel, this is a curved bevel 1817 to provide better tracking of the needle. This feature is important for a straight needle, but particularly important for a pre-shaped needle to prevent the needle tip from accidentally puncturing the balloon from being aligned away from the opening of the groove of the balloon.

A second feature to prevent any mis-alignment of the needle involves marking the shafts of the balloon catheter, the guide wire and the needle. First is to align the balloon groove to the exit port opening of the guide wire. This is accomplished by having a marker on the proximal shaft of the balloon in an OTW system aligned with the balloon groove and another marker on the proximal shaft of the guide wire aligned with the exit port opening of the guide wire. When these two markers line up during use, the exit port is positioned toward the opening of the balloon groove. If the drug injection needle is straight, there will be no need to align the needle with the guide wire, but if the needle is pre-shaped, a marker can be placed on the proximal shaft of the needle or placed in such a way that to line up with the marker on the guide wire to ensure that the curved needle will exit the port opening at an appropriate angle without puncturing the balloon. This feature will also be described in subsequent paragraphs.

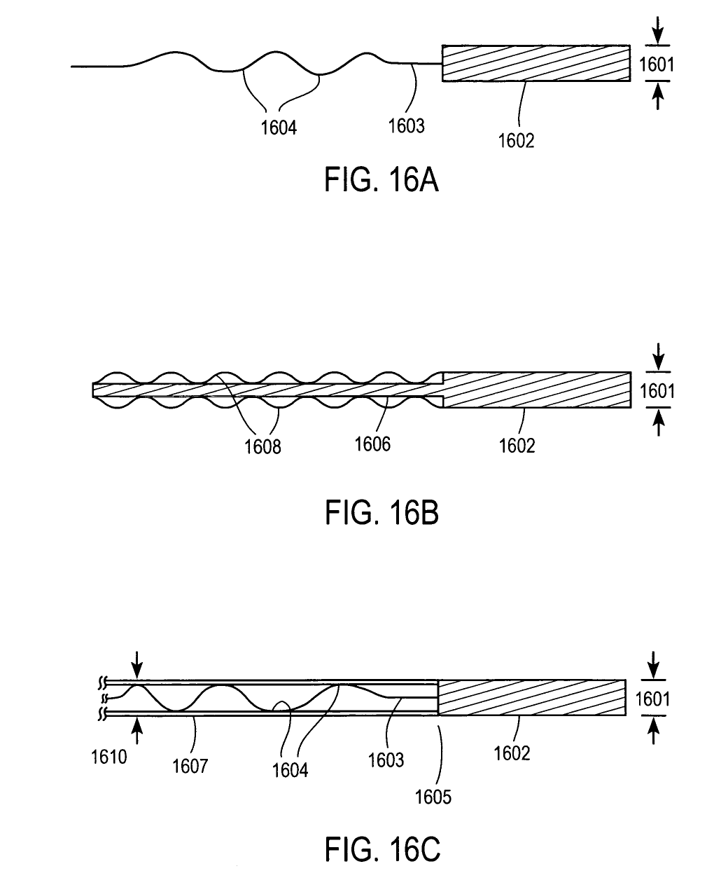

Further, in an embodiment where the guide wire is an individual and separate unit from both the stent catheter delivery system and the drug injection needle, a proximal plug is employed to facilitate back loading of the guide wire into the stent delivery catheter system. FIGS. 16A to 16C describe the proximal plug and its combination with the modified hollow guide wire. The objective of having a proximal plug is to prevent blood seeping from the needle exit port opening on the distal tip of the modified hollow guide wire back through the hollow guide wire lumen to the proximal end of the modified hollow guide wire while the guide wire is used for access and to guide the stent delivery system to the target vessel. One important requirement of the proximal plug is to have the outer diameter of the plug 1601 no larger than the diameter 1610 of the guide wire, as illustrated in FIGS. 16A to 16C, because during the procedure, the guide wire is back loaded into the stent delivery system. Back loading is the preferred mode of practice for users because the proximal end of the guide wire is much stiffer than the distal floppy tip. Back loading prevents damage to the fragile distal tip and is much easier to thread through the guide wire lumen because of its stiffness. Since the proximal end of the modified guide wire has to pass through the guide wire lumen and the proximal plug is to be attached to the proximal end, the outer diameter of the plug should not be larger than the outer diameter of the guide wire so that it can enter and pass through the guide wire lumen of the stent catheter delivery system unobstructed. FIG. 16A shows one embodiment of the proximal plug with the body 1602 and a tail portion comprising a shaft 1603 shaped like a wave-form 1604. The body 1602 has a diameter 1601 no larger than the outer diameter of the guide wire to which it is inserted and can be made of polymer or metal. The body should have a low coefficient of friction and/or able to be coated with a material of low friction such as Teflon™, microglide, or similar coatings to facilitate movement. The shaft 1603 is a metallic wire where the wave-form 1604 shape. This wave-form 1604 shape acts as a spring when inserted into the guide wire lumen and exerts a force normal to the side wall of the guide wire, generating friction within the guide wire lumen to prevent the plug disengaging from the guide wire. The length of the shaft 1603 and the wave-form 1604 is arbitrary, but should be sufficiently long enough to allow the wave-form 1604 to generate sufficient friction so the plug does not slide out easily. FIG. 15B shows an alternate design of a proximal plug to the guide wire where a body 1602 has a similar outer diameter 1601 as the outer diameter of the guide wire. The shaft 1608 extends from the body 1602 but has a smaller outer diameter than 1601. A spring or a high friction material 1606 attached to the shaft with the same idea to generate friction between the shaft and the inner wall of the guide wire lumen to prevent the plug disengagement. FIG. 16C shows a proximal plug of FIG. 16A integrated into a modified hollow guide wire as described. The outer diameter of the proximal plug 1601 is flush with the outer diameter 1610 of the guide wire with a smooth transition at the gap junction 1605 between the body of the plug and the guide wire. The wave forms 1604 is compressed and exerts a force firmly against the side wall 1607 of the modified guide wire.

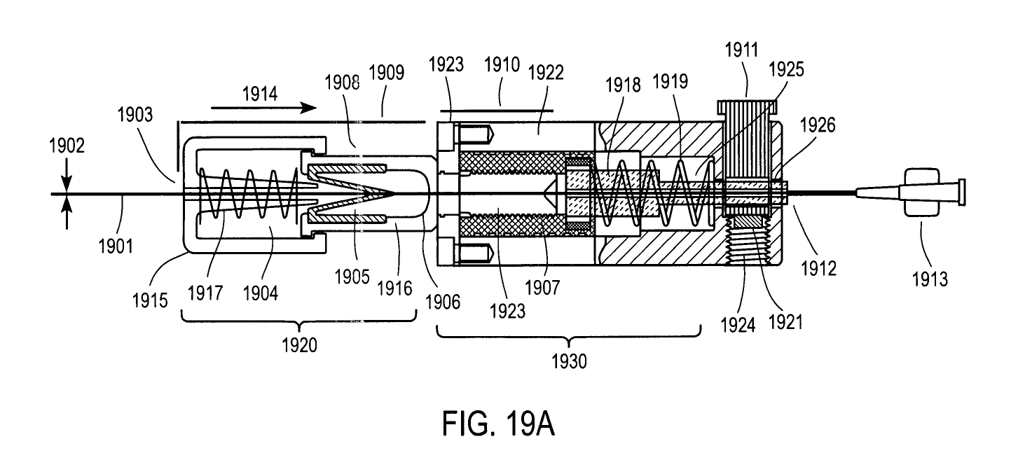

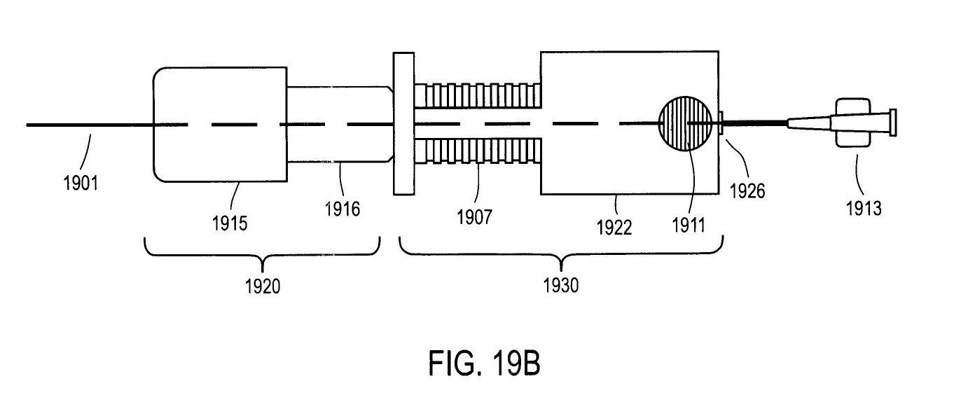

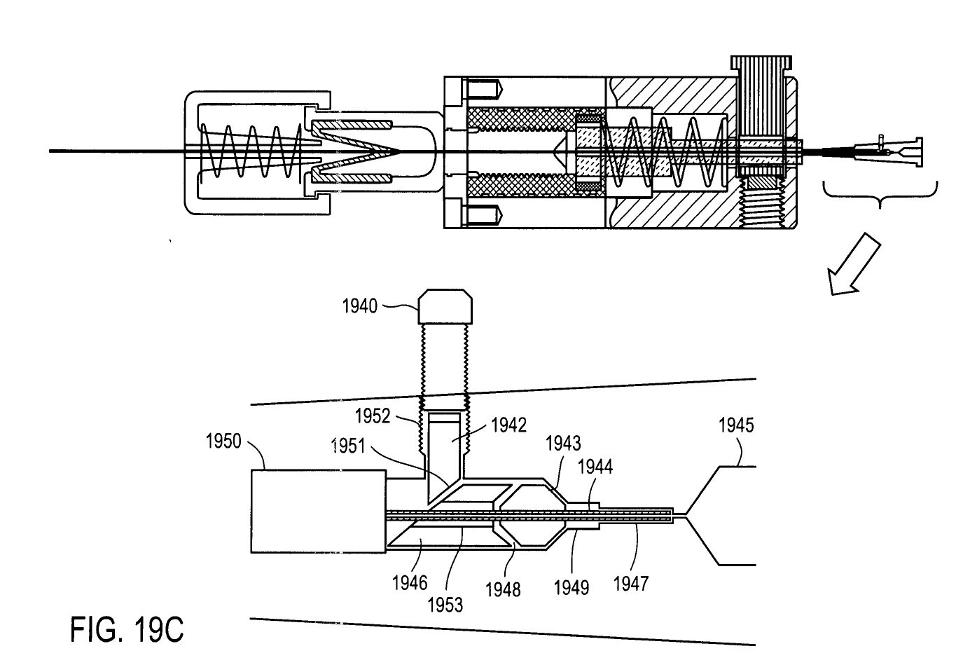

The needle, as mentioned above, may be straight or pre-shaped. An important characteristic of the needle is that it must be flexible and compliant so that it can bend easily when advanced out of the exit port at the registration point inside the guide wire lumen. Any material, metal or polymer, with super-elasticity or shape memory is suitable. Nickel-titanium (NiTinol) is a logical alloy of choice for this application. FIGS. 19A and 19B illustrates various parts comprising the entire needle system including the needle body 1901, the connection mechanism 1920 to the guide wire and the control mechanism of the drug delivery needle 1930. The outer diameter 1902 of the injection needle is uniform along the entire length of the needle. The sidewall (not shown) of the needle is uniform along its entire body except for the tip where it is beveled to a sharp point for ease of tissue penetration. The proximal end of the needle shaft is attached to a luer 1913 which is used to connect to a drug reservoir for drug injection. The entire body will front load through both the connection mechanism 1920 and the control mechanism 1930 via the tip of the needle as described.

Now the connection between the guide wire (not shown) and the connection mechanism 1920 will be described. The purpose of this connection mechanism 1920 is to provide a firm attachment to fix the guide wire relative to the control mechanism 1930 during the procedure. It further functions to prevent blood from leaking from the guide wire lumen and also helps to identify the relative rotational and translational position of the guide wire with respect to the needle (and its position relative to the balloon groove, indirectly through the position of the guide wire). The connection mechanism 1920 comprises a push cap 1915 pushed over a valve casing 1916 which contains an elastomer seal 1905 to hold the guide wire in place. The spring 1904 inside the push cap functions to hold the cap in position allowing the valve to remain close under normal circumstances. A cylindrical member 1917 inside and an integral part of the push cap 1915 is used to force open the elastomer seal 1905 as the spring 1904 is compressed in the direction 1914. As the user pushes the guide wire through the opening 1903 of the connection mechanism into the connection mechanism 1920, it is suggested that there be at least one marking on the guide wire to indicate the radial orientation of the exit port opening on the wire, as well as when the guide wire proximal end is near the back wall 1906 of the connection mechanism. Such marking on the guide wire can be aligned with a marking represented by the line 1909 on the connection mechanism 1920. The connection mechanism 1920 is not fixed to the needle 1901, but since it is fixed with respect to the guide wire (not shown), it can be aligned with a marking represented by the line 1910 on the control mechanism 1930 which is fixed with respect to the needle to ensure alignment of the guide wire and the needle. Therefore, once the guide wire is firmly attached to the connection mechanism 1920 with the markings on the guide wire aligned to the marking on the connection mechanism 1920 and the control mechanism 1930, the tip of the needle will be at a known position relative to the exit port opening of the guide wire.

An alternate embodiment can have a needle pre-loaded within the hollow guide wire. In this embodiment, rather than having the luer permanently fixed to the proximal end of the drug injection needle as in the last embodiment, the luer, which is used to connect to the drug reservoir for injection, will be separately attached to the end of the drug injection needle. The length of the injection needle will be relatively longer than the length of the guide wire to leave room for attaching to the connection mechanism and control mechanism as well as the detachable luer for drug infusion. Compared to an injection needle with a permanently fixed proximal luer, which requires front loading the connection mechanism and control mechanism through the distal tip of the injection needle before loading into the proximal end of the guide wire lumen, this embodiment will back load into the connection mechanism and the control mechanism through the proximal end of the injection needle before attaching to the detachable luer.

FIG. 19C describes the embodiment of a detachable luer. The detachable luer is typically made of a hard plastic casing with a proximal end that attaches to a drug reservoir and a distal end that inserts into the control mechanism. The distal end of the detachable luer has a hard plastic or nylon sleeve that has several functions. First, it provides support for the injection needle 1944; second, it protects the needle and provides a point of fixture when inserted into the control mechanism to be anchored between the side hole of the lock bushing and the through hole in the housing wall; third, it guides the needle 1944 into the securing mechanism inside the detachable luer where the injection needle is fixed.

Several components within the detachable luer function together to secure the needle in place. Central to the securing mechanism is an elastomer O-ring 1943. Distal to the O-ring 1943 but proximal to the sleeve 1950 is a wedge block 1946 with a lumen 1953 which is slightly larger than the outer diameter of the needle. On the other side, proximal to the O-ring 1943 is a chamber 1949 which narrows proximally into a tunnel 1947. The chamber 1949 has an inner diameter larger than the outer diameter of the injection needle 1944 but similar to that of the inner lumen 1953 of the wedge block 1946. On the contrary, the tunnel has an inner diameter similar to the outer diameter of the injection needle so it results in a tight fit with the needle. The tunnel narrows at its proximal end slightly to provide a stop for the needle and leads into the proximal luer opening 1945 which connects to the drug reservoir for drug infusion. Two other components, a screw 1940 and a beveled piston 1942, in conjunction with the wedge block 1946, the O-ring 1943 and the chamber 1949 function together to secure the injection needle. The beveled piston 1942 is positioned below the screw such that when the screw 1940 is being turned, it will push the beveled piston 1942 down toward and make contact with the wedge block 1946. Since the piston is beveled and matches the slanted surface of the wedge block 1946 at contact, as the piston is being forced down, the wedge block is pushed to the right and proximal which in turns squeeze the O-ring 1943 between the wedge block 1946 and the chamber 1649. By virtue of the shape of the O-ring 1943, tapered into both the inner lumen 1953 of the wedge block 1946 and into the chamber 1949, the O-ring 1943 squeezes and secures the needle in place as the screw 1940 tightens down on the beveled piston 1942. It is to be understood that the shape of the O-ring 1943 only need to be tapered on its ends, as shown in the figure, to squeeze onto the needle to secure it in place without deforming the needle, it can take on any shape or even be made of a different material than elastomer. Further, it should be understood that the beveled piston 1942 is split and has a groove in the middle (not shown) so a needle 1944 can pass through. As the beveled piston 1942 is pushed down onto the wedge block 1946, this split or groove prevents any interference with or deformation of the needle 1944 as the beveled piston 1942 is pushed further downward to squeeze the O-ring 1942 via the wedge block 1946.

It should also be understood that a marking can be placed on the body of the needle to line up and match with another marking on the detachable luer casing for the purpose of aligning the needle, if pre-shaped, so that the operator knows the direction of the needle curvature. These two markings can also align with the markings proposed for the connection mechanism and control mechanism (as described later) for overall system alignment. Further, this marking on the needle can inform the user if the proximal end of the needle is backed up all the way into the tunnel 1947 to ensure a fully secured fit before drug infusion.

Movement of the injection needle relative to the guide wire is achieved via the control mechanism 1930. The connection mechanism 1920 is coupled to the base plate 1923 of the control mechanism 1930. Inside the control mechanism 1930 contains a needle stop mechanism and a needle lock mechanism. The former controls the position in which the needle can advance and the latter locks and release the needle to allow for movement. The control mechanism 1930 comprises a housing 1922 containing a needle stop adjustment mechanism and a needle lock assembly. The needle stop adjustment mechanism located in the distal compartment within the connection mechanism 1930 comprises the components such as a needle stop adjuster dial 1907, a threaded stem 1923, a spring 1919, and a needle assembly holder 1918. The spring 1919 functions to provide a compression force on the needle stop adjustment dial 1907 so that once the needle stop location is set, the dial does not accidentally turn by itself without manual actuation from the operator. As the operator turns the needle stop adjuster dial 1907, its position changes along the longitudinal axis and its proximal surface acts as a stopper to control the position in which the needle assembly holder 1918 can be advanced forward. The needle lock mechanism is located in the proximal compartment of the control mechanism and is used to lock and release the needle for movement. This mechanism comprises a spring 1921, a spring retainer 1924 and a lock bushing 1911 that includes a side hole 1912. The proximal shaft 1925 of the needle assembly holder 1918 runs through the side hole 1912 of the lock bushing 1911 and a through hole 1926 in the proximal wall of the housing 1922. The spring retainer 1924 is threaded into a matching hole in the housing to hold the lock spring 1921 in compression. By adjusting the position of the spring retainer 1924 in the hole of the housing, the compression force generated on the lock spring 1921 varies. The lock spring 1921 exerts an upward force on the lock bushing 1911 causing the side hole 1912 to move off alignment with respect to the proximal through hole 1926 of the housing. This misalignment causes the proximal shaft 1925 to be locked within the side hole 1912 of the lock bushing 1911 and the through hole 1926 of the housing. This locking mechanism is used to hold the needle in place, whether the needle is within the guide wire or advance into the tissue for drug injection. Free movement of the needle is accomplished only when the lock bushing 1911 is pressed down to allow alignment of the side hole 1912 and the through hole 1926. In the case of a pre-shaped needle, a square shaped proximal shaft 1925 along with a square shaped side hole 1912 and through hole 1926 may be desired to prevent undesired rotation of the needle. In a modified embodiment, the coupling of the control mechanism 1930 to the connection mechanism 1920 can incorporate a mechanism to permit a limited degree of rotation perhaps with a lock or via friction so that the operator can control the rotation of the shaped needle tip during the procedure.

FIG. 19B shows an exterior view of the needle assembly with the needle 1901 threaded through the connection mechanism 1920 and the control mechanism 1930. The push cap 1915 and the valve housing 1916 of the connection mechanism 1920 are visible. The needle stop adjuster dial 1907, the control mechanism housing 1922, the lock bushing 1911 and the through hole 1926 are visible. A needle 1901 is threaded through both the connection mechanism 1920 and the control mechanism 1930 with a luer 1913 attached to its proximal end.

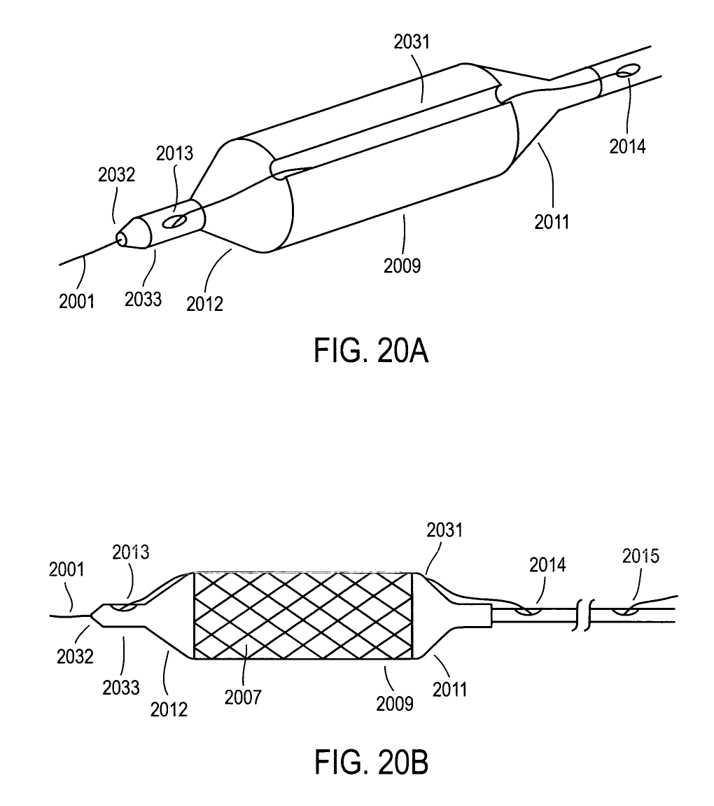

FIGS. 20A to 20B illustrate one embodiment of the uniquely molded balloon integrated in a stent delivery catheter system. The catheter delivery system to deliver and deploy a stent is generally similar to current catheter delivery system used in the coronary system. The catheter can be over-the-wire or rapid-exchange in its manner of interaction with the guide wire. The catheter system, in application to the coronary system, should be compatible with the diameter of the guide wire used in a typical procedure, preferably a 0.014″ diameter guide wire. The proximal shaft and proximal member of the catheter system is generally similar. The major difference with this catheter delivery system is the uniquely shaped balloon and the manner in which the balloon is uniquely extruded into a “horse-shoe” like shape where there is a groove or trench on the outside profile of the balloon for the guide wire to provide support to the balloon and for access of the drug injection needle to the vessel wall through the exit port on the guide wire.

FIG. 20A shows a three dimensional view of the embodiment (without a stent) where the wire exits the guide wire lumen on the proximal shaft of the stent delivery catheter through port opening 2014, through the groove 2031 along the length of the balloon, into the distal guide wire lumen opening 2013 on the distal member 2033 of the balloon 2009 and out through the distal tip 2032 of the catheter system. Note that in this embodiment, the groove is not displayed along the balloon shoulder regions of 2011 and 2012. Alternatively, the balloon may be extruded such that a groove can extend from the proximal shoulder region 2011, along the length of the balloon, to the distal shoulder region 2012 of the balloon thereby provide added security and guidance to the movement of the guide wire over the balloon when accessing a lesion.

FIG. 20B shows a side view of the embodiment with the balloon inflated and a stent expanded. This embodiment illustrates a rapid exchange (RX) system where the guide wire enters the proximal shaft through opening 2015 exits proximal member of the balloon through lumen 2014 and rides in the balloon groove 2031 along the outer profile of the balloon 2009 entering the lumen 2013 of the distal balloon member and exiting the distal tip 2032 of the balloon catheter. Alternatively, the delivery catheter can be an over-the-wire system (OTW) where the wire enters the proximal end of the guiding catheter (not shown) instead of from a lumen on the proximal shaft.

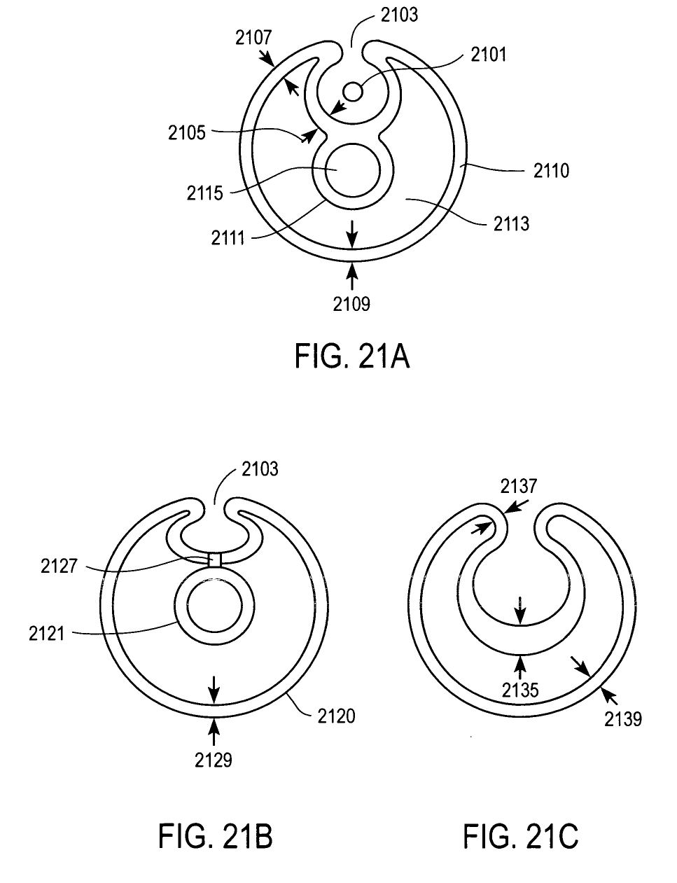

FIGS. 21A to 21C show the general cross-sectional shape of the various extruded embodiments of the balloon. FIG. 21A shows a preferred extruded embodiment of the balloon. The balloon has a round and generally circular shaped outer profile except for a groove or trench 2103 to fit the guide wire 2101 once it is inflated. In this embodiment, the balloon can be extruded by inserting a mandrel or a pin in the center of the balloon during extrusion, thus creating an inner lumen 2115 for the inner member 2111 that may also serve as an inflation lumen for the balloon. This inner lumen or inflation lumen is approximately in the order of about 0.006″ to about 0.020″ in diameter. The thickness of the balloon wall 2110 can be made uniform, but in this embodiment, it varies. For instance, balloon wall at thickness 2109 is the thinnest and likely in the approximate range of about 0.0005″ to about 0.003″; balloon wall at thickness 2107 is approximately in the range of about 0.003″ to about 0.006″ whereas balloon wall thickness 2105 is approximately in the range of about 0.004″ to 0.008″. The purpose of the varying thickness of balloon wall is to accommodate the different stresses that the balloon wall experiences during inflation and provide adequate support at the curve where the groove or trench is located. It is particularly important to have a thick balloon wall at 2105 and 2107 which are responsible for maintaining the groove in the balloon. When the balloon is blown in the molding process to form the desired shape after inflation, the balloon will be blown as illustrated with an inner member taking the place of the pin in the center of the balloon where holes can be made for inflation. The inner member is likely connected to a proximal member and the lumen will become an inflation lumen where contrast or dye material is injected through the inflation lumen to inflate the balloon under fluoroscopy.

FIG. 21B illustrates an alternate embodiment of an extruded balloon. In this embodiment, the balloon 2129 is uniform around the circumference. However, a part of the balloon wall is unique in that the material at the bottom of the groove 2103 is attached and fused via a junction 2127 with the outer diameter material of the supporting member 2121 either continuously or at least at several points along the length of the support member 2121. This can help reduce the need for a thickened balloon wall to overcome the stress and strain as observed during balloon inflation to maintain the shape of the groove. Similar to the previous embodiment, without attaching the balloon wall 2120 to the supporting member, the groove can collapse or disappear during balloon inflation unless the volume and pressure of the inflation is controlled with a degree of precision to which it is impractical to implement. Both the balloon extrusion designs in FIGS. 21A and 21B help to reduce balloon wall thickness and thus may help in improving overall balloon/catheter profile and deliverability. Similarly, Pebax 63-D is one of many balloon materials that has a sufficient balloon compliance which can be used in such designs.

FIG. 21C shows a balloon extruded into its shape without a pin in its center in the extrusion process. The balloon is extruded without a mandrel in the center and since the material is not attached to the center for support, the balloon wall thickness need to vary similar to and perhaps even to a further extent than that in FIG. 21A in order to overcome the stresses during inflation to maintain the desired shape. In particular, balloon wall 2135 is approximately in the range of about 0.010″ to 0.015″, balloon wall thickness 2137 is approximately in the range of about 0.006″ to 0.010″ and balloon wall thickness 2139 is approximately in the range of about 0.001″ to 0.005″. Unless the proper material is used in this configuration, the balloon/catheter profile and deliverability may be sacrificed. During the blowing process, the proximal and distal portions of the balloon will be bonded to a proximal and distal member. Inside the proximal member, there will be an inflation lumen created for contrast injection to inflate the balloon.

The procedure of stent delivery, deployment and drug injection is now described. In the same embodiment of the guide wire described above, the needle is not in the guide wire during the guide wire's initial placement and engagement with any catheter. Both the needle and the guide wire are separate units but combinable in application. Prior to use, the uniquely shaped balloon is deflated and folded onto the stent delivery system with a stent (with or without drug coating) crimped and mounted over it. A long wire or mandrel is preloaded in the system in place of the guide wire, from the tip of the catheter system, into a portion of the distal member, along the groove or trench of the balloon, and into the proximal member, as shown in FIG. 20A or 20B (which illustrates a guide wire with an inflated balloon instead of a mandrel with a deflated and crimped balloon). Before the procedure, the user will remove the mandrel and flushes the guide wire lumen or path with heparinized saline. Similarly, the inner lumen of the modified hollow guide wire is flushed with heparinized saline and then with the proximal plug inserted into the proximal end of the guide wire. The guide wire, with the proximal plug in place, will be back loaded onto the stent delivery catheter system from the catheter's distal tip. The guiding catheter is expected to be placed in the proper anatomy when the combined stent delivery catheter system and the guide wire is inserted into the guiding catheter. Like a typical interventional procedure, the stent delivery system and the wire is delivered via the guide catheter to the target anatomy. In the case of the heart and a coronary vessel, the guide catheter is cannulated to the proximal section of the target coronary artery through the aortic arch, the guide wire will be advanced distally into the coronary artery, cross the lesion and positioned so that the stent delivery catheter system can ride over the guide wire to the target site.

Before inflation of the balloon and deployment of the stent, the proximal plug on the guide wire is removed and the needle is inserted into the modified hollow guide wire from its proximal end. The needle lumen would similarly have been flushed with heparinized saline or pre-filled with drug prior to insertion into the guide wire to prevent any blood from clotting inside the lumen while it is being advanced inside the guide wire lumen. As the needle is advanced towards the tip of the modified hollow guide wire, the connection mechanism on the proximal portion of the needle shaft will approach the proximal end of the guide wire. Once the connection mechanism attaches to the proximal end of the guide wire, the needle tip is positioned near the exit port at the distal portion of the guide wire.

Once positioned at the target site, the balloon is inflated and the stent is deployed with the needle positioned inside the guide wire. After the stent is deployed, the balloon remains inflated in place and the needle is advanced using the control mechanism proximal to inject drug. As described previously, the needle can be advanced to a desired depth depending on the treatment objective. The depth of penetration will be controlled by the translation control mechanism at the proximal end of the needle. Similarly, the needle, if pre-shaped, can rotate to an angle, independently controlled by the rotational control mechanism, so that drug can be injected at multiple locations without moving the guide wire and its exit port. However, if drug is to be injected at multiple locations along the vessel, the balloon must remain inflated, and the needle retracted, before repositioning the guide wire exit port can translate along the groove over the length of the balloon for multiple drug injections. Drug injections via the drug delivery needle can be performed both within the stented region as well as distal or proximal to the stented region. If the region proximal or distal to the stent region is targeted, the balloon needs to be deflated, re-positioned and reinflated again before injection can take place. The number of injections and the amount of drug to be injected depend on a range of factors including but not limited to the objective of intervention, the target vessel, the type of lesion, the drug to be used and how well the patient's heart can tolerate having the balloon inflated for drug injections without resulting in arrhythmias, even though one of the advantages of a balloon with a groove is so that blood can flow through the groove to the distal region of the artery to supply oxygen and minimize ischemic injury.

In this combination, the injection of drug into the tissue through the needle inside the modified guide wire is preferably accomplished in conjunction with the specially molded balloon. However, the drug delivery through the injection needle and hollow guide wire combination is not limited to use only with the specially molded balloon, but to any another structure that can position the guide wire close to the vessel wall while providing support to the needle as it penetrates the vessel wall. In this embodiment, the needle 1706 and guide wire 1701 utilize the inflated balloon 1709 to position the guide wire 1701 at a specific location and use the balloon as support for the needle 1706 to penetrate the tissue. In the application of this combination therapy, a user can rotate the balloon to the approximate target regions along the vessel wall to selectively inject drug using the needle from the guide wire.

Similar to the drug delivery balloon catheter with a retractable needle previously described in this disclosure, the use of a drug delivery guide wire, as described, relies on the inflation of a uniquely shaped balloon as support for the needle to penetrate the vessel wall to inject drug. A conventional balloon of uniform circular circumference and diameter fails to provide the necessary “groove” and support for the drug delivery guide wire-needle combination. Further, an inflated balloon will occlude the vessel cutting off blood and oxygen supply to the distal portion of the artery. Leaving the balloon inflated and occluding the vessel for too long may potentially lead to ischemia of the distal vasculature and is a potential problem with conventional balloons. The current therapy of using a grooved balloon and the guide wire-needle drug injection combination provides the advantage of at least allowing a limited supply of blood and oxygen to flow into the distal artery through the groove and further allow drug injection through the guide wire lumen where the drug injection needle is housed, to infuse drug distally if necessary. Therefore, even if a large amount of drug injection is required, risk of ischemic injury to the vessel can be minimized. As for multiple drug injections at various locations along the vessel, consecutive deflation and re-inflation of the balloon which may lead to excessive trauma to the vessel or over-disruption of the lesion or plaque along the vessel wall may also be minimized because the guide wire can be repositioned within the balloon groove for needle injections. Alternatively, this same concept of a grooved balloon can incorporate multiple trenches or grooves to maximize blood flow distally to the lesion during drug injection. While this may provide more blood and oxygen flow distal to the lesion and the balloon can be left inflated longer without leading to ischemic injury, drug injections proximal and distal to the lesion will still require multiple inflation and deflation of the balloon in order to position the guide wire and injection needle. Furthermore, the balloon is required to have either thickened walls or the balloon wall/material may need to be bonded or joined to the outer diameter of the inner member to gain sufficient strength to overcome the stress during inflation.

Finally, all the combination therapies described in this application may incorporate the delivery of drug using a carrier which may be more “friendly” to the vessel or target tissue, and thus absorbed more readily by the tissue than the drug without the carrier. Furthermore, nanoparticles may accompany direct infusion of drug dissolved or suspended in carrier fluid. The nanoparticles and the carrier fluid together are referred to as the infusate. Similar to drugs, the infusate parameters can be optimized to include a mixed population of multiple types of drugs within the nanoparticles. Optimization can also be further based on the size distribution of nanoparticles, the bulk property of the nanoparticles, the surface chemistry of nanoparticles, host-material response property of the nanoparticle and the carrier, and the rheological property of the carrier, such that the nanoparticles will complement and enhance the absorption of drugs. The nanoparticles themselves can be modified to provide the physician an option for point-of-care selection to be commensurate with the patient. The benefit of using nanoparticles in a combination therapy is to target the most complete biologically active distribution of the therapeutic agent transluminally into an adventitial space, distally in the target vessel, in the side branches of bifurcation lesions or deep into the myocardial tissue. Tailoring the nanoparticles with a particular surface chemistry and property can facilitate transport of the drug or therapeutic agent through the arterial wall and the different layers of tissue thus attaining the most effective and efficient absorption deep into the myocardium.

For example, the infusate payload can be a solution or controlled release suspension of a drug, small molecule, peptide, protein, or gene in microspheres, nanospheres or liposomes to address regional disease. Another form of an active agent to be used in this therapy is apolipoprotein A1 (also known as Apo A-1), its variance and mimetics. The microspheres can be made of poly(lactic-co-glycolic acid) (PLGA) or other materials. Drugs such as everolimus [lower case if generic name], sirolimus, paclitaxel, statins, oxidant signal antagonists (e.g., Atherogenics AGI-1067), other anti-inflammatories, e-NOS regulators (e.g., Aventis), and tissue inhibitor of matrix metalloproteinases (TIMMPS), etc. can be also be incorporated into the infusate. For example, the aforementioned drugs may be delivered in the catheter via multiple different drug formulations made of liposome, nanoparticle, lipid coated microbubble along with the other microparticles described above.

Overall, the use of nanoparticles and carriers to deliver drug therapy into a vessel can provide a physician with various different options in conjunction with the different embodiments described, such as bathing of the coronary vasculature, retrograde perfusion, and local drug injections. As would be understood to those skilled in the art, specific formulations can be made to those drugs used in accordance with the present invention according to methods known in the art.

In the foregoing specification, the invention has been described with reference to specific exemplary embodiments thereof. It will, however, be evident that various modifications and changes may be made thereto without departing from the broader spirit and scope of the invention as set forth in the appended claims. The specification and drawings are, accordingly, to be regarded in an illustrative rather than a restrictive sense.

What is claimed is:

1. A method to treat an arterial region comprising:
advancing percutaneously a drug eluting stent system into an arterial vessel to a target site;
inflating a balloon on the drug eluting stent system to deliver a drug eluting stent coated with a first drug at the target site;
advancing percutaneously a retrograde perfusion delivery catheter system to a venous region distal to the coronary artery from a venous vessel;
inflating a second balloon on the retrograde perfusion delivery catheter to occlude the venous vessel;
perfusing with pressure in retrograde a second drug from the retrograde perfusion delivery catheter from a venous region into capillaries; and
bathing the second drug in the capillaries as the retrograde pressure forces the second drug into the capillaries and tissue surrounding the arterial region.

2. The method as in claim 1 wherein the arterial region is a diseased coronary artery with a vulnerable plaque lesion.

3. The method as in claim 2 wherein the drug eluting stent contains a drug comprising at least one drug selected from the group consisting of sirolimus, everolimus, paclitaxel, anti-restenotic agents, anti-inflammatory agents, and anti-proliferative agents.

4. The method as in claim 1 wherein the drug eluting stent system comprises a drug delivery port to deliver at least one of a drug solution and a plaque stabilizing agent.

5. The method as in claim 1 wherein the second drug is different from the first drug on the drug eluting stent, and wherein said second drug is complementary to or augments the effectiveness of the first drug on the drug eluting stent.

6. The method of claim 1 wherein said second drug comprises at least one of a drug selected from the group consisting of a small molecule drug, peptide or gene construct in solution, a controlled release microsphere formulation, a liposome formulation, a second drug formulation and a therapeutic agent.

7. The method as in claim 1 wherein the second balloon on the retrograde perfusion delivery catheter is a compliant balloon that will not injure the venous vessel.

8. An apparatus comprising:
a percutaneous retrograde perfusion drug delivery catheter to treat a vulnerable plaque lesion in a diseased arterial region from a venous system in conjunction with a catheter with an inflatable balloon inflated proximal to the vulnerable plaque lesion, the percutaneous retrograde perfusion drug delivery catheter having three lumens with a first lumen for inflation of a balloon, a second lumen for passing of a guidewire, and a third lumen for pressurized retrograde delivery of a drug from the venous system in a direction of the arterial region.

9. The apparatus as in claim 8 wherein the perfusion drug delivery catheter further comprises a drug eluting stent system and the inflatable balloon is inflated after delivering said drug eluting stent at or near the vulnerable plaque lesion in the diseased arterial region.

10. The apparatus as in claim 9 wherein the drug eluting stent is coated with one or more of a second drug selected from a group consisting of sirolimus, everolimus, paclitaxel, anti-restenotic agents, anti-proliferative agents, and anti-inflammatory agents.

11. The apparatus as in claim 10 wherein the first drug comprises a therapeutic agent having a formulation that is different from the second drug coated onto a drug eluting stent, wherein said first drug is complementary to or augments effectiveness of the second drug.

12. The apparatus as in claim 9 wherein the diseased arterial region also comprises diffuse disease and a discrete lesion.

13. The apparatus as in claim 12 wherein the apparatus is used to treat the arterial region from a venous vessel by way of pressurized retrograde perfusion to bathe the first drug into capillaries and tissue surrounding the diseased arterial region.

14. The apparatus as in claim 8 wherein the first drug comprises at least one of a drug selected from the group consisting of small molecule drugs, peptide constructs in a solution, gene constructs in a solution, controlled release microsphere formulations and liposome formulations.

15. The apparatus as in claim 8 wherein the catheter system is delivered into a coronary venous vessel downstream of a target coronary arterial region and a coronary capillary bed in relation to normal blood flow.

16. The apparatus of claim 15 wherein said coronary venous vessel is the coronary sinus.

17. The apparatus as in claim 15 wherein the pressurized retrograde delivery infuses the first drug from the coronary sinus into the coronary capillary bed and myocardial tissue surrounding the target coronary arterial region.

18. The apparatus as in claim 8 wherein the apparatus is used in conjunction with a drug delivery device other than a drug eluting stent.

19. The apparatus of claim 18 wherein said drug delivery device is a balloon catheter incorporating a needle for drug delivery and a molded balloon inflatable catheter using a flexible and retractable needle in a modified or hollowed guide wire for drug delivery.

20. The apparatus of claim 19 wherein said balloon contains a groove along the balloon region for receiving said guide wire.

21. An apparatus comprising:
a percutaneous drug eluting stent coated with a first drug, wherein said stent is mounted over a catheter delivery system with a flexible needle that can be manually advanced from the catheter along a proximal balloon shoulder into a vessel wall at an angle for injection of a second drug when the balloon is inflated after the drug eluting stent is deployed.

22. The apparatus as in claim 21 wherein the first drug comprises at least one of a drug selected from the group consisting of sirolimus, everolimus, paclitaxel, anti-restenotic agents, anti-inflammatory agents and anti-proliferative agents.

23. The apparatus as in claim 22 wherein the second drug comprises a formulation that is different from the first drug, wherein said second drug is complementary to or augments effectiveness of the first drug.

24. The apparatus as in claim 21 wherein the catheter delivery system is advanced into a diseased coronary artery comprising at least one of discrete lesions, diffusion lesions and vulnerable plaque.

25. The apparatus as in claim 21 wherein the needle is pre-shaped and emerges from the catheter delivery system in a curved fashion to puncture the vessel wall.

26. The apparatus of claim 25 wherein the needle comprises a hollow center.

27. The apparatus of claim 25 wherein the needle is made of a shape memory alloy or polymer.

28. The apparatus as in claim 25 wherein the needle only enters the vessel wall at an angle similar to that of the shoulder of the balloon relative to the vessel wall after the balloon is inflated.

29. The apparatus as in claim 28 wherein the needle is advanced into a range of depths of a coronary artery ranging from the internal elastic lamina to the periadventitial space depending on the drug to be delivered and the state of the diseased artery.

30. The apparatus as in claim 21 wherein the catheter delivery system can be used to simultaneously deploy the drug eluting stent and infuse the second drug in a coronary artery.

31. The apparatus as in claim 21 wherein the catheter delivery system is used to first deploy the drug eluting stent and then deflate the balloon to advance the catheter delivery system along different positions of a coronary artery for multiple infusions of the second drug.

32. The apparatus as in claim 21 wherein the second drug is injected into the periadventitial space of a coronary artery and migrates through a fat layer to bathe the epicardium and other coronary arteries.

33. A method to treat a vessel with a vulnerable plaque comprising:
advancing percutaneously a drug eluting stent to a target site in the vessel, wherein said drug eluting stent is coated with a first drug, and wherein said drug eluting stent is mounted on a needle catheter system;
inflating a balloon on the needle catheter system to deploy the drug eluting stent;
advancing a flexible needle inside a lumen along a shoulder of the inflated balloon;
extracting the flexible needle from an exit port on the shoulder of the inflated balloon into the vessel wall;
injecting a second drug into the vessel wall; and
bathing the second drug in the vessel wall for absorption by the vessel.

34. The method as in claim 33 wherein the first drug on the drug eluting stent comprises at least one of a drug selected from the group consisting of sirolimus, everolimus, paclitaxel, anti-restenotic agents, anti-inflammatory agents and anti-proliferative agents.

35. The method as in claim 33 wherein the second drug comprises a formulation that is different from the first drug on the drug eluting stent, wherein said second drug is complementary to or augments effectiveness of the first drug.

36. The method as in claim 33 wherein the vessel with a vulnerable plaque is a coronary artery with discrete lesions or diffuse disease.

37. The method as in claim 33 wherein the needle is flexible and pre-shaped and emerges from the catheter delivery system in a curved fashion to puncture the vessel wall.

38. The method of claim 37 wherein the needle comprises a hollow center.

39. The method of claim 37 wherein the needle is made of a shape memory alloy or polymer.

40. The method as in claim 37 wherein the needle only enters the vessel wall at an angle similar to that of the shoulder of the balloon relative to the vessel wall after the balloon is inflated.

41. The method as in claim 40 wherein the needle is advanced into a range of depths in a coronary artery ranging from the internal elastic lamina to the periadventitial space depending on the drug to be delivered and state of the disease.

42. The method as in claim 33 wherein the second drug is injected into the periadventitial space of a coronary artery and migrates through a fat layer to bathe the epicardium and other coronary arteries.

43. The method as in claim 33 wherein the catheter delivery system can be used to first deploy the drug eluting stent and then deflate the balloon to advance the catheter delivery system along different positions of a coronary artery for multiple infusions of the second drug.

44. An apparatus comprising:
a guidewire having a hollow center, said guidewire comprising a retractable, flexible needle to inject a first drug to treat a vessel with a vulnerable plaque; and
a stent catheter system comprising a drug eluting stent coated with a second drug and a balloon molded with an external groove or trench on an outer profile along a length of the balloon, wherein said stent is mounted on said balloon and wherein said groove or trench provides free guidewire movement along the balloon and allows free needle access to a vessel wall from the guidewire after the balloon is inflated against the vessel wall.

45. The apparatus as in claim 44 wherein the second drug comprises at least one of a drug selected from the group consisting of sirolimus, everolimus, paclitaxel, anti-restenotic agents, anti-inflammatory agents and anti-proliferative agents.

46. The apparatus as in claim 45 wherein the first drug comprises a therapeutic agent having a formulation that is different from the second drug on the drug eluting stent, wherein said first drug is complementary to or augments effectiveness of the second drug in treatment of the vessel.

47. The apparatus as in claim 44 wherein the vessel is a diseased coronary artery with discrete lesions and/or diffuse disease.

48. The apparatus as in claim 44 wherein the needle is pre-shaped and emerges from the catheter delivery system in a curved fashion to puncture the vessel wall.

49. The apparatus as in claim 48 wherein the needle comprises a hollow center.

50. The apparatus of claim 48 wherein the needle is made of a shape memory alloy or polymer.

51. The apparatus as in claim 48 wherein the needle is advanced into a range of depths in a coronary artery ranging from internal elastic lamina to periadventitial space depending on the drug to be delivered and disease state of the coronary artery.

52. The apparatus as in claim 44 wherein the catheter delivery system is used to simultaneously deploy the drug eluting stent and infuse the first drug.

53. The apparatus as in claim 36 wherein the stent catheter delivery system is used to first deploy the drug eluting stent, and then deflate the balloon to advance the catheter delivery system along different positions of a coronary artery to delivery drug using the delivery guide wire.

54. The apparatus as in claim 44 wherein once the drug to be delivered is injected into a periadventitial space of a coronary artery, the drug will migrate through a fat layer to bathe the epicardium and other coronary arteries.

55. A method to treat a vessel comprising:
advancing percutaneously a stent catheter system to a vessel with a vulnerable plaque said catheter system comprising a drug eluting stent coated with a first drug and a balloon molded with an external groove or trench on an outer profile along a length of the balloon, wherein said stent is mounted on said balloon; inflating the balloon to deploy the drug eluting stent against the vessel wall;
positioning a needle exit port on a guidewire at a selected region against the vessel wall along the external groove of the balloon;
advancing a flexible and retractable needle inside the guidewire out of the needle exit port;
penetrating the flexible needle into the vessel wall;
injecting a second drug into the vessel wall; and
bathing the second drug in the vessel wall for absorption by the vessel.

56. The method as in claim 55 wherein a first drug on the drug eluting stent comprises at least one of a drug selected from the group consisting of sirolimus, everolimus, paclitaxel, anti-restenotic agents, anti-inflammatory agents and anti-proliferative agents.

57. The method as in claim 56 wherein the second drug comprises a formulation that is different from the first drug, wherein said second drug is complementary to or augments the first drug in treating vulnerable plaque.

58. The method as in claim 55 wherein the catheter delivery system is advanced into a diseased coronary artery with discrete lesions and/or diffusion lesions.

59. The method as in claim 55 wherein the needle is flexible and pre-shaped and emerges from the catheter delivery system in a curved fashion to puncture the vessel wall.

60. The method of claim 59 wherein the needle comprises a hollow center.

61. The method of claim 59 wherein the needle is made of a shape memory alloy or polymer.

62. The method as in claim 59 wherein the needle is advanced into a range of depths in a coronary ranging from the internal elastic lamina to the periadventitial space depending on the second drug to be delivered and disease state of the artery.

63. The method as in claim 55 wherein the second drug is injected into the periadventitial space in a coronary artery and migrates through a fat layer to bathe the epicardium and other coronary arteries.

64. The method to treat a diseased vessel comprising:
deploying a drug eluting stent at a target site in the diseased vessel with a vulnerable plaque, wherein said stent is coated with a first drug;
delivering an infusate to circulate downstream of the target site;
bathing a vessel region with the infusate to allow for diffusion into the vasculature;
wherein the overall therapeutic effect on the diseased vessel is augmented by the combination therapy provided by the first drug and the complementary effects of the infusate acting within the vasculature via diffusion.

65. The method as in claim 64 wherein the diseased vessel comprises a diseased artery that includes a vulnerable plaque.

66. The method as in claim 64 wherein the first drug comprises at least one of a drug selected from the group consisting of sirolimus, everolimus, paclitaxel, anti-proliferative agents, anti-inflammatory agents, and anti-restenotic agents.

67. The method as in claim 64 wherein the infusate comprises a carrier fluid together with nanoparticles such as liposomes.

68. The method as in claim 67 wherein the infusate parameters are optimized according to the disease state based on at least one of vulnerable plaque, diffused diseases and discrete lesion found in the vessel.

69. The method as in claim 67 wherein the infusate parameters are optimized by selecting one or more of a mixed population of drugs, nanoparticle size distribution, bulk property of the nanoparticle, surface chemistry of the nanoparticle, host-material response property of the nanoparticles, and Theological property of the carrier.

70. The method as in claim 64 wherein the bathing of a vessel region is accomplished by retrograde perfusion or access into a pericardial sac.

71. The method as in claim 64 wherein the infusate comprises a second drug comprising at least one of a solution or a controlled release suspension to address the vessel disease.

72. The method as in claim 71 wherein the solution or the controlled release suspension has at least one agent selected from a group consisting of proteins, peptides and genes encapsulated in particles.

73. The method as in claim 71 wherein the solution or the controlled release suspension contains an agent selected from a group consisting of liposomes, statins, anti-inflammatory agents, e-NOS regulators, and oxidant signal antagonists.
